(19) United States
(12) Patent Application Publication
(10) Pub. No.: US 2023/0313152 A1
(43) Pub. Date: Oct. 5, 2023

(54) C-TERMINAL TRUNCATED GDE FOR THE TREATMENT OF GLYCOGEN STORAGE DISEASE III

(71) Applicants: GENETHON, EVRY-COURCOURONNES (FR); INSERM (INSTITUT NATIONAL DE LA SANTÉ ET DE LA RECHERCHE MÉDICALE), PARIS (FR); UNIVERSITE D'EVRY VAL D'ESSONNE, EVRY-COURCOURONNES (FR)

(72) Inventors: GIUSEPPE RONZITTI, FONTAINEBLEAU (FR); PATRICE VIDAL, BOUSSY SAINT-ANTOINE (FR)

(21) Appl. No.: 18/022,534

(22) PCT Filed: Aug. 23, 2021
(86) PCT No.: PCT/EP2021/073309

§ 371 Date: Feb. 22, 2023

(30) Foreign Application Priority Data
Aug. 24, 2020 (EP) 20192377.8

(51) Int. Cl. C12N 9/10; C12N 15/86; A61P 3/00

(52) U.S. Cl. CPC C12N 9/1051; C12Y 204/01025; C12N 15/86; A61P 3/00; C12N 2750/14143

(57) ABSTRACT

The present invention relates to a functional C-terminal truncated GDE polypeptide for the treatment of glycogen storage disease III.

The present invention relates to the treatment of glycogen storage disease III (GSDIII).

### BACKGROUND OF THE INVENTION

Mutations in the AGL gene cause genetic deficiency of glycogen debranching enzyme (GDE), or “amylo-alpha-1,6-glucosidase, 4-alpha-glucanotransferase”, an enzyme involved in glycogen degradation. GDE has two independent catalytic activities which occur at different sites on the protein: a 4-alpha-glucotransferase activity and an amylo-1,6-glucosidase activity. Genetic deficiency of GDE causes an incomplete glycogenolysis in glycogen storage disease III (GSDIII), resulting in accumulation of abnormal glycogen with short outer chain in various organs, mostly liver and muscle. The disease is characterized by hepatomegaly, hypoglycemia, short stature, variable myopathy and cardiomyopathy. Most patients have GSDIII involving both liver and muscle (type Ma), while some patients (˜15 percent) have only liver involvement (type IIIb). Liver symptoms normally occur in childhood. Liver cirrhosis and hepatocellular carcinoma have been reported in some cases (Chen et al., 2009, Scriver's Online Metabolic & Molecular Bases of inherited Disease, New York: McGraw-Hill; Kishnani et al., 2010, Genet Med 12, 446-463). Muscle weakness could be present during childhood. It becomes more prevalent in adults with onset in the third or fourth decade. There is significant morbidity from progressive muscle weakness and patients in later stages can become wheel chair bound. Patients can also develop cardiomyopathy. There is significant clinical variability in the severity of the symptoms that these patients develop. The progressive myopathy and/or cardiomyopathy and/or peripheral neuropathy are major causes of morbidity in adults (Kishnani et al., 2010, Genet Med 12, 446-463; Cornelio et al., 1984, Arch Neurol 41, 1027-1032; Coleman et al., 1992, Ann Intern Med 116, 896-900). Reports of possible neurological manifestations associated with the disease derive from clinicians working with GSDIII patients, who reported attention fluctuations, deficiencies in executive functions and impaired emotional skills (Michon et al., 2015, J Inherit Metab Dis, 38(3): 573-580). Accordingly, in the GDE−/− mouse model of the disease, an extensive accumulation of glycogen throughout the nervous system was documented (Pagliarani et al., 2014, Biochim Biophys Acta, 1842(11): 2318-2328; Liu et al., 2014, Mol Genet Metab, 111(4): 467-476) although a careful characterization of the phenotype associated with the accumulation of glycogen is still missing. Current treatment is symptomatic, and there is no effective therapy for the disease. Hypoglycemia can be controlled by frequent meals high in carbohydrates with cornstarch supplements or nocturnal gastric drip feedings. Patients with myopathy have been treated with a diet high in protein during the daytime plus overnight enteral infusion. In some patients transient improvement in symptoms has been documented, but there are no systemic studies or long-term data demonstrating that the high protein diet prevents or treats the progressive myopathy (Kishnani et al., 2010, Genet Med 12, 446-463). These approaches do little to alter the long term course and morbidity of these diseases.

Therefore, there is still a need for a long-term treatment of GSDIII. Gene therapy aiming to stably replace the GDE protein in the affected tissues appears as a potential therapeutic approach. However, the large size of the GDE transgene constitutes a major impediment since it cannot fit the size limit of most gene therapy vectors. Indeed, the human AGL gene is 85 kb in length and composed of 35 exons, encoding a 7.4-kb mRNA that includes a 4596-bp coding region and a 2371-bp 3′ untranslated sequence to express a 175 kDa GDE protein (Bao Y et al., 1996, Genomics., 38(2):155-65). This constitutes a real issue since the minimum size of a GDE expression cassette (including for example at least a promoter, the GDE coding sequence, a polyA signal and the two ITRs for an AAV vector) would be larger than 5 kb, the genome size limit that can be packaged into an AAV gene therapy vector for in vivo gene delivery. The inventors have previously proposed the use of dual AAV vectors to overcome this size limitation. Following this approach, two vectors, each containing a portion of the large transgene coding sequence, are used to transduce the same cell. Although the use of dual AAV vectors is promising, it would be preferable to provide a gene therapy strategy implementing only one viral vector for both economic and practical reasons. In patent application WO 2020/030661, the present inventors described another approach based on the use of a “mini GDE polypeptide” that fits in a single viral vector. Yet, alternatives to the mini GDE polypeptides previously described in WO 2020/030661 are desirable.

### SUMMARY OF THE INVENTION

The present invention relates to a functional truncated GDE polypeptide, wherein said functional truncated GDE polypeptide comprises a C-terminal deletion of at least 1 amino acid, such as at least 5, 10, 15, 20, 25, 30, 35, 40, 45, 50, 55, 60, 65, 70, 75, 80, 85, 90, 95, 100, 105 or at least 110 amino acids and at most 112 amino acids with respect to a reference functional full-length human GDE sequence, and wherein the functional truncated GDE polypeptide does not comprise the sequence as shown in SEQ ID NO:59.

In a particular embodiment, the reference functional full-length human GDE has an amino acid sequence as shown in SEQ ID NO:1, SEQ ID NO:2, SEQ ID NO:3, SEQ ID NO:61, SEQ ID NO:62 or SEQ ID NO:63, or has an amino acid sequence having at least 80, 85, 90, 95, 96, 97, 98 or at least 99 percent sequence identity to SEQ ID NO:1, SEQ ID NO:2, SEQ ID NO:3, SEQ ID NO:61, SEQ ID NO:62 or SEQ ID NO:63.

In a particular embodiment:
(i) the reference functional full-length human GDE sequence has an amino acid sequence as shown in SEQ ID NO:1 or SEQ ID NO:61, and said truncated GDE polypeptide is deleted of at least one amino acid and of at most 112 amino acids selected from amino acids at positions 1421 to 1532 with respect to SEQ ID NO:1 or SEQ ID NO:61;
(ii) the reference functional full-length human GDE sequence has an amino acid sequence as shown in SEQ ID NO:2 or SEQ ID NO:62, and said truncated GDE polypeptide is deleted of at least one amino acid and of at most 112 amino acids selected from amino acids at positions 1404 to 1515 with respect to SEQ ID NO:2 or SEQ ID NO:62; or
(iii) the reference functional full-length human GDE sequence has an amino acid sequence as shown in SEQ ID NO:3 or SEQ ID NO:63, and said truncated GDE polypeptide is deleted of at least one amino acid and of at most 112 amino acids selected from amino acids at positions 1405 to 1516 with respect to SEQ ID NO:3 or SEQ ID NO:63.

In a further particular embodiment:
(i) the reference functional full-length human GDE sequence has an amino acid sequence as shown in SEQ ID NO:1 or SEQ ID NO:61, and said truncated GDE polypeptide is deleted of at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61;
(ii) the reference functional full-length human GDE sequence has an amino acid sequence as shown in SEQ ID NO:2 or SEQ ID NO:62, and said truncated GDE polypeptide is deleted of at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62; or
(iii) the reference functional full-length human GDE sequence has an amino acid sequence as shown in SEQ ID NO:3 or SEQ ID NO:63, and said truncated GDE polypeptide is deleted of at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63.

In a particular embodiment, the functional truncated GDE polypeptide further comprises a deletion or a combination of deletions with respect to SEQ ID NO:1, SEQ ID NO:2, SEQ ID NO:3, SEQ ID NO:61, SEQ ID NO:62 or SEQ ID NO:63, wherein the deletion(s) is(are) selected from any deletion referred to as Δ1, Δ2, Δ3, Δ4, Δ5, Δ6, Δ7, Δ8, Δ9, Δ10, Δ11, Δ12, and Δ13 in table 2, in particular Δ9, Δ10, Δ11, Δ12, and Δ13.

In a particular embodiment, the functional truncated GDE polypeptide has an amino acid sequence as shown in SEQ ID NO:7-14, SEQ ID NO:25-33 or SEQ ID NO:64-66, or has an amino acid sequence having at least 80, 85, 90, 95, 96, 97, 98 or at least 99 percent sequence identity to SEQ ID NO:7-14, SEQ ID NO:25-33 or SEQ ID NO:64-66.

In a further particular embodiment, the functional truncated GDE polypeptide has an amino acid sequence selected from SEQ ID NO:7, SEQ ID NO:8, SEQ ID NO:9, SEQ ID NO:64, SEQ ID NO:65, or SEQ ID NO:66, in particular SEQ ID NO:7, SEQ ID NO:8 or SEQ ID NO:9, in particular SEQ ID NO:7 or has an amino acid sequence having at least 80, 85, 90, 95, 96, 97, 98 or at least 99 percent sequence identity to SEQ ID NO:7, SEQ ID NO:8, SEQ ID NO:9, SEQ ID NO:64, SEQ ID NO:65, or SEQ ID NO:66, in particular to SEQ ID NO:7, SEQ ID NO:8 or SEQ ID NO:9, in particular to SEQ ID NO:7.

The present invention also relates to a nucleic acid molecule encoding the functional truncated GDE polypeptide as defined above.

A further aspect to the invention relates to an expression cassette, comprising, preferably in this order:
a promoter;
optionally, an intron;
the nucleic acid molecule encoding the functional truncated GDE polypeptide as defined above; and
a polyadenylation signal.

The present invention also relates to a vector, in particular a viral vector such as an AAV vector, comprising the nucleic acid molecule or the expression cassette as described above.

The present invention also relates to an isolated cell transformed with the nucleic acid molecule, the expression cassette or the vector as defined above, wherein the cell is in particular a liver cell, a muscle cell, a cardiac cell or CNS cell.

Another aspect of the invention relates to the functional truncated GDE polypeptide, the nucleic acid molecule, the expression cassette, the vector, or the cell as defined above, for use as a medicament.

The invention also relates to the functional truncated GDE polypeptide, the nucleic acid molecule, the expression cassette, the vector, or the cell as defined above, for use in a method for treating a disease caused by a mutation in the AGL gene encoding GDE.

In a further particular embodiment, the invention relates to the functional truncated GDE polypeptide, the nucleic acid molecule, the expression cassette, the vector, or the cell as defined above, for use in a method for treating GSDIII (Cori disease).

### LEGENDS TO THE FIGURES

FIG. 1. C-terminal deleted ΔCter-1-GDE has similar efficacy than full-size GDE in the rescue of muscle strength in GSDIII mice. GDE knockout (KO) mice were injected with PBS (KO, PBS) or with 1×10¹² vg/mouse of an AAV vector expressing C-terminal truncated GDE (KO, ΔCter-1 or full-size GDE (KO, FS). PBS-injected wild-type mice were used as controls (WT, PBS). Wire-hang performance was measured one week before (D-7) or 5 weeks after (D35) vectors injection and expressed as falls per minute. Statistical analysis was performed by two-way ANOVA (*=p<0.05 vs. WT, PBS; #=p<0.05 vs. KO, PBS; n=3-5 per group).

FIG. 2. C-terminal deleted ΔCter-1-GDE has comparable efficacy to full-size GDE in the clearance of glycogen in heart and quadriceps of GSDIII mice. GDE knockout (KO) mice were injected with PBS (KO, PBS) or with 1×10¹² vg/mouse of an AAV vector expressing C-terminal truncated GDE (KO, ΔCter-1) or full-size GDE (KO, FS). PBS-injected wild-type mice were used as controls (WT, PBS). Five weeks after vector injection, animals were sacrificed and glycogen accumulation was measured in heart and quadriceps. Statistical analyses was performed by ANOVA (*=p<0.05 vs. WT, PBS; #=p<0.05 vs. KO, PBS; n=3 per group).

FIG. 3. C-terminal deleted ΔCter-1-GDE has similar efficacy than full-size GDE in the rescue of muscle strength in GSDIII mice, 3 months post-injection. GDE knockout (KO) mice were injected with PBS (KO_PBS); with 3.3×10¹² vg/mouse or 9.9 Δ10¹² vg/mouse (High dose) of an AAV vector expressing C-terminal truncated GDE (ΔCter-1); or with 3.3 Δ10¹² vg/mouse of an AAV vector expressing full-size GDE (GDE FS). PBS-injected wild-type mice were used as controls (WT_PBS). Wire-hang performance was measured one week before (FIG. 3.A) and 3 months after (FIG. 3.B) vectors injection and expressed as falls per minute. Statistical analysis was performed by two-way ANOVA (n=3-5 per group).

FIG. 4. Western blot revealing the presence of GDE in the heart, diaphragm, triceps and quadriceps. GDE knockout (KO) mice were injected with PBS (KO_PBS); with 3.3 Δ10¹² vg/mouse or 9.9×10¹² vg/mouse (High dose) of an AAV vector expressing C-terminal truncated GDE (ΔCter-1); or with 3.3 Δ10¹² vg/mouse of an AAV vector expressing full-size GDE (GDE FS). PBS-injected wild-type mice were used as controls (WT_PBS). The different constructions injected into Agl^{-/-} mice are denoted ΔCter-1, ΔCter-1 High dose and GDE FS. KO mice injected with PBS were used as negative controls.

### DETAILED DESCRIPTION OF THE INVENTION

Despite the lack of knowledge regarding the three-dimensional structure of the GDE protein, the present inventors have identified a C-terminal truncated GDE polypeptide whose coding sequence is small enough to be packaged into a gene therapy vector, in particular into a single AAV vector, while preserving the GDE functionality.

The present invention thus relates to a functional C-terminal truncated GDE polypeptide. This polypeptide can advantageously be used in a method for treating a disease caused by a mutation in the AGL gene encoding GDE, in particular in a method for treating GSDIII (Cori disease).

1—C-Terminal Truncated GDE Polypeptide

The C-terminal truncated GDE polypeptide according to the present invention is a functional GDE polypeptide whose coding sequence is small enough to be efficiently packaged into a gene therapy vector, in particular into a single AAV vector.

By “functional” GDE polypeptide is meant a polypeptide that retains, at least in part, at least one of the enzymatic activities of the GDE protein, preferably all of the enzymatic activities of the GDE protein. As a consequence, the functional GDE polypeptide implemented in the present invention is able to rescue glycogen accumulation and muscle strength in vivo. As defined above, GDE enzymatic activities are a 4-alpha-glucotransferase activity and an amylo-1,6-glucosidase activity, involved in glycogen degradation. The transferase activity of GDE relocates three glucose units of glycogen from one chain to another. This leaves one glucose unit at the branch point, which is subsequently released as glucose by the glucosidase activity. In a particular embodiment, the functional GDE polypeptide of the invention has the same functionality as a full-length GDE polypeptide, in particular as a full-length human GDE polypeptide. For example, a functional GDE polypeptide of the invention may have an activity of at least 50%, 60%, 70%, 80%, 90%, 95%, or at least 99% in relation to one, preferably both, enzymatic activities described above, or at least 100%, as compared to a full-length human GDE protein, in particular the full-length human GDE protein of SEQ ID NO:1, SEQ ID NO:2, SEQ ID NO:3, SEQ ID NO:61, SEQ ID NO:62, or SEQ ID NO:63. The activity of the GDE protein of the invention may even be of more than 100%, such as of more than 110%, 120%, 130%, 140%, 150%, 200%, 500%, 700%, or even more than 1000% of the activity of a full-length human GDE protein, in particular the full-length human GDE protein of SEQ ID NO:1, SEQ ID NO:2, SEQ ID NO:3, SEQ ID NO:61, SEQ ID NO:62, or SEQ ID NO:63. In a particular embodiment, the functional GDE polypeptide of the invention has the same functionality as a full-length GDE polypeptide in particular as a full-length human GDE polypeptide in muscular tissues such as heart or quadriceps. For example, a functional GDE polypeptide of the invention may have an activity in muscular tissues such as heart or quadriceps of at least 50%, 60%, 70%, 80%, 90%, 95%, or at least 99% in relation to one, preferably both, enzymatic activities described above, or at least 100%, as compared to a full-length human GDE protein, in particular the full-length human GDE protein of SEQ ID NO:1, SEQ ID NO:2, SEQ ID NO:3, SEQ ID NO:61, SEQ ID NO:62, or SEQ ID NO:63. The activity in muscular tissues such as heart or quadriceps of the GDE protein of the invention may even be of more than 100%, such as of more than 110%, 120%, 130%, 140%, 150%, 200%, 500%, 700%, or even more than 1000% of the activity of a full-length human GDE protein, in particular the full-length human GDE protein of SEQ ID NO:1, SEQ ID NO:2, SEQ ID NO:3, SEQ ID NO:61, SEQ ID NO:62, or SEQ ID NO:63.

By “functional” GDE polypeptide is meant a GDE polypeptide that is not pathological. In particular, the functional GDE polypeptide of the invention is not a GDE polypeptide found in a patient with GSDIII (Cori disease), such as GSDIIIa or GSDIIIb.

A skilled person is readily able to determine whether a polypeptide is a functional GDE polypeptide. Suitable methods would be apparent to those skilled in the art. For example, one suitable in vitro method involves inserting a nucleic acid encoding a polypeptide into a vector, such as a plasmid or viral vector, transfecting or transducing host cells, such as 293T or HeLa cells, or other cells such as Huh7, with the vector, and assaying for GDE activity. Suitable methods are described in more details in the experimental part below. For example, GDE activity may be determined by measuring the glucose produced after incubating homogenized mouse tissues with limit dextrin. Other methods include testing the GDE activity by determining GDE expression in tissues of a GDE KO animal, such as by western-blot, by following the glucose produced from glycogen phosphorylase-digested glycogen, by evaluating muscle strength of treated GDE-KO animals by wire-hang after administration of the vectors, such as after one, two or three months after administration, or by evaluating the rescue of glycogen accumulation in muscle and/or cardiac tissue.

By “C-terminal truncated GDE polypeptide” is meant a GDE polypeptide that is truncated at the C-terminal end with respect to a reference full-length GDE sequence. In a particular embodiment, the “C-terminal end” corresponds to a region consisting of the last 112 amino acids, i.e. consisting of the most C-terminal 112 amino acids of said reference full-length human GDE sequence.

In a particular embodiment, the C-terminal truncated GDE polypeptide is a functional truncated human GDE polypeptide, which is truncated with respect to a reference full-length human GDE sequence. The term “truncated human GDE polypeptide” encompasses any human GDE polypeptide that is rendered shorter by amino acid deletion, with respect to a reference full-length human GDE sequence from which the truncated human GDE is derived. In particular, the C-terminal truncated human GDE polypeptide is deleted of at least 1 amino acid at the C-terminal end with respect to a reference full-length human GDE sequence. Preferably, the C-terminal truncated human GDE polypeptide is deleted of at least about 10, 20, 30, 40, 50, 60, 75, 90, 100, or at least 110 and at most about 112 amino acids at the C-terminal end with respect to a reference full-length human GDE sequence. In a preferred embodiment, the functional truncated human GDE polypeptide is deleted of at least about 50, 60, 65, 70, 80, 90 or 100 amino acids at the C-terminal end with respect to a reference full-length human GDE sequence.

In a particular embodiment, the C-terminal truncated GDE polypeptide is deleted of 1 to 112 consecutive amino acids at the C-terminal end with respect to a reference full-length human GDE sequence, wherein the 1 to 112 deleted amino acids are the most C-terminal amino acids of said reference full-length human GDE sequence.

In particular, the C-terminal truncated GDE polypeptide may be deleted of at least 5, 10, 15, 20, 25, 30, 35, 40, 45, 50, 55, 60, 65, 70, 75, 80, 85, 90, 95, 100, 105, or at least 110 and at most 112 consecutive amino acids at the C-terminal end with respect to a reference full-length human GDE sequence, wherein the deleted amino acids are the most C-terminal amino acids of said reference full-length human GDE sequence. For example, the C-terminal truncated GDE polypeptide may be deleted of 5, 10, 15, 20, 25, 30, 35, 40, 45, 50, 55, 60, 65, 70, 75, 80, 85, 90, 95, 100, 105, 110 or 112 consecutive amino acids at the C-terminal end with respect to a reference full-length human GDE sequence, wherein the deleted amino acids are the most C-terminal amino acids of said reference full-length human GDE sequence.

For the sake of clarity, a “C-terminal truncated GDE polypeptide deleted of 5 amino acids, wherein the deleted amino acids are the most C-terminal amino acids” corresponds to a polypeptide truncated of 5 consecutive amino acids starting from the C-terminal end, i.e. amino acids 1, 2, 3, 4, et 5, starting from the C-terminal end.

As used herein with respect to any disclosed values or ranges, the term “about” indicates that the stated numerical value allows for slight imprecision, e.g., reasonably close to the value or nearly, such as plus or minus 10%, in particular such as plus or minus 5%, of the stated values or ranges.

As further described below, the C-terminal truncated GDE polypeptide according to the invention is at least truncated at the C-terminal end with respect to a reference full-length GDE sequence, but may also be truncated at other part(s), with respect to the reference full-length GDE sequence. Accordingly, the C-terminal truncated GDE polypeptide may be deleted of at least about 10, 20, 30, 40, 50, 60, 75, 90, 100, 125, 150, 175, 190, 200, 225, 250, 275, 300, 325, 350, 375, 400, 425, 450, 475, 500 or at least about 525 amino acids with respect to a reference full-length human GDE sequence. In a preferred embodiment, the C-terminal truncated GDE polypeptide is deleted of at least about 50, 100 or 150 amino acids with respect to a reference full-length human GDE sequence.

In a preferred embodiment, the size of the functional C-terminal truncated GDE polypeptide is small enough so that the nucleic acid molecule encoding said GDE polypeptide can be packaged into an AAV vector with suitable control sequences. Preferably, the size of the functional C-terminal truncated GDE polypeptide is less than 1480 amino acids, such as less than 1475, 1470, 1465, 1460, 1455, 1450, 1445 or less than 1440 amino acids. Preferably the size of the C-terminal truncated GDE polypeptide is less than 1472 amino acids.

In a particular embodiment, the size of the functional C-terminal truncated GDE polypeptide is of at least 650 amino acids, such as at least 700, or at least 750 amino acids.

In a particular embodiment, the size of the functional C-terminal truncated GDE polypeptide is of at least 650 amino acids and at most 1480 amino acids. In a particular embodiment, the size of the functional C-terminal truncated GDE polypeptide is of at least 700 amino acids and at most 1472 amino acids.

In a particular embodiment, the C-terminal truncated GDE polypeptide that is truncated with respect to a reference full-length human GDE sequence may comprise one or more additional amino acid modifications with respect to said reference full-length human GDE sequence. In particular, in addition to the deletion(s) that are further described below, the functional truncated GDE polypeptide may comprise one or more amino acid modifications such as amino acid insertion, deletion and/or substitution as compared to the reference full-length human GDE sequence. For example, the functional truncated GDE polypeptide may comprise from 1 to 10 (e.g. 1, 2, 3, 4, 5, 6, 7, 8, 9 or 10) additional amino acid modifications, in particular from 1 to 5 (e.g. 1, 2, 3, 4 or 5) additional amino acid modifications, as long as the functionality of the truncated GDE polypeptide is preserved. In a particular embodiment, when the C-terminal truncated GDE polypeptide further comprises a N-terminal deletion, a methionine can be added at the N-terminal end.

In the context of the present invention, “a reference full-length human GDE sequence” does not encode a pathological GDE polypeptide. In particular, the reference full-length human GDE sequence does not encode a pathological variant found in a patient with GSDIII, comprising mutation(s), deletion(s) or insertion(s) as compared to the wild-type non-pathological full-length human GDE sequence.

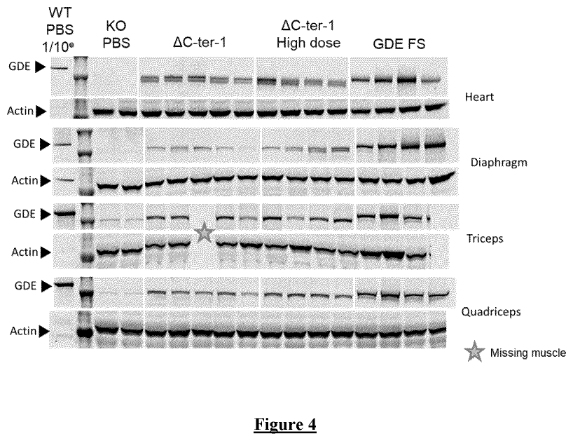

For example, Okubo et al. (Hum Genet (1998) 102:1-5) describe a pathological GDE polypeptide comprising 15 amino acids (as shown in SEQ ID NO:59) different from the wild type control which instead comprises a sequence as shown in SEQ ID NO:60 (see FIG. 4 of Okubo et al.).

Contrary to the disclosure of Okubo et al., the “reference full-length human GDE sequence” as described in the present invention comprises the sequence as shown in SEQ ID NO:60. Thus, the “reference full-length human GDE sequence” as described in the present invention does not comprise the sequence as shown in SEQ ID NO:59. Consequently, the C-terminal truncated GDE polypeptide of the invention does not comprise the sequence as shown in SEQ ID NO:59.

In the context of the present invention, “a reference full-length human GDE sequence” encompasses all native isoforms of human GDE. Bao and colleagues (Genomics, 1997, 38, 155-165) identified the presence of six transcript variants encoding for three GDE protein isoforms. Transcript variants 1-4 encode for the same protein, namely GDE isoform 1. Transcript variants 5 and 6 encode for GDE isoforms 5 and 6 respectively.

The term “reference full-length human GDE polypeptide” thus encompasses all native isoforms of human GDE including the precursor form, as well as modified or mutated by insertion(s), deletion (s) and/or substitution(s) GDE proteins or fragments thereof that are functional derivatives of GDE. In particular, the reference full-length human GDE sequence is selected from the group consisting of SEQ ID NO:61 (corresponding to wild-type GDE isoform 1, UniProtKB identifier: P35573-1), SEQ ID NO:1 (corresponding to a variant of GDE isoform 1), SEQ ID NO:62 (corresponding to wild-type GDE isoform 5, UniProtKB identifier: P35573-2), SEQ ID NO:2 (corresponding to a variant of GDE isoform 5), SEQ ID NO:63 (corresponding to wild-type GDE isoform 6, UniProtKB identifier: P35573-3), and SEQ ID NO:3 (corresponding to a variant of GDE isoform 6).

In a particular embodiment, the reference full-length human GDE has at least 80, 85, 90, 95, 96, 97, 98 or at least 99 percent sequence identity to SEQ ID NO:1, SEQ ID NO:2, SEQ ID NO:3, SEQ ID NO:61, SEQ ID NO:62, or SEQ ID NO:63, in particular to SEQ ID NO:1, SEQ ID NO:2 or SEQ ID NO:3. In a particular embodiment, the reference full length human GDE has at least 80, 85, 90, 95, 96, 97, 98 or at least 99 percent sequence identity to SEQ ID NO:1 or SEQ ID NO:61 and has the same length in terms of number of amino acids as SEQ ID NO:1 or SEQ ID NO:61. In a particular embodiment, the reference full length human GDE has at least 80, 85, 90, 95, 96, 97, 98 or at least 99 percent sequence identity to SEQ ID NO:2 or SEQ ID NO:62 and has the same length in terms of number of amino acids as SEQ ID NO:2 or SEQ ID NO:62. In a particular embodiment, the reference full length human GDE has at least 80, 85, 90, 95, 96, 97, 98 or at least 99 percent sequence identity to SEQ ID NO:3 or SEQ ID NO:63 and has the same length in terms of number of amino acids as SEQ ID NO:3 or SEQ ID NO:63.

In a particular embodiment, the reference full-length human GDE sequence has an amino acid sequence as shown in SEQ ID NO:1 or SEQ ID NO:61, in particular SEQ ID NO:1 which correspond to GDE isoform 1.

The C-terminal truncated GDE polypeptide of the invention comprises a C-terminal deletion of at least one amino acid and of at most 112 amino acids selected from amino acids at positions 1421 to 1532 with respect to SEQ ID NO:1 or SEQ ID NO:61. In a particular embodiment, the C-terminal truncated GDE polypeptide comprises a C-terminal deletion of at least 1, 5, 10, 15, 20, 25, 30, 35, 40, 45, 50, 55, 60, 65, 70, 75, 80, 85, 90, 95, 100, 105, or at least 110 amino acids selected from amino acids at positions 1421 to 1532 with respect to SEQ ID NO:1 or SEQ ID NO:61.

In a particular embodiment, the C-terminal truncated GDE polypeptide comprises a C-terminal deletion of at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61.

In a preferred embodiment, the C-terminal truncated GDE polypeptide comprises a C-terminal deletion of 112 consecutive amino acids from amino acids at positions 1421 to 1532 with respect to SEQ ID NO:1 or SEQ ID NO:61. For the sake of clarity, in this embodiment, the deletion relates to the deletion of all consecutive amino acids in the mentioned range of positions. This C-terminal deletion of 112 consecutive amino acids from amino acids at positions 1421 to 1532 with respect to SEQ ID NO:1 or SEQ ID NO:61 is hereafter called the deletion “ΔCter-1”.

In a particular embodiment, the functional C-terminal truncated GDE polypeptide of the invention which comprises a C-terminal deletion as described above, is further deleted of at least one amino acid with respect to SEQ ID NO:1 or SEQ ID NO:61, wherein the deleted amino acid(s) is at least one amino acid at positions 1-428, 668-769, 895-1087 and/or 1195-1232 with respect to SEQ ID NO:1 or SEQ ID NO:61. In a further particular embodiment, the functional C-terminal truncated GDE polypeptide of the invention which comprises a C-terminal deletion as described above, is further deleted of at least about 10, 20, 30, 40, 50, 60, 75, 90, 100, 125, 150, 175, 190, 200, 225, 250, 275, 300, 325, 350, 375, 400, 425, 450, 475, 500 or at least about 525 amino acids, wherein the deleted amino acid(s) are selected from any amino acids at positions 1-428, 668-769, 895-1087, and/or 1195-1232 with respect to SEQ ID NO:1 or SEQ ID NO:61. In this embodiment, the deleted amino acids may be consecutive amino acids or non-consecutive amino acids, as long as they are selected from any amino acids at positions 1-428, 668-769, 895-1087 and/or 1195-1232 with respect to SEQ ID NO:1 or SEQ ID NO:61.

In a particular embodiment, the functional C-terminal truncated GDE polypeptide of the invention comprises at least the amino acid residues at positions 429-666, 770-892, 1088-1194, 1235-1420 with respect to SEQ ID NO:1 or SEQ ID NO:61.

In a particular embodiment, the functional C-terminal truncated GDE polypeptide of the invention comprises at least the amino acid residues at positions 429-667, 770-894, 1088-1194, 1233-1420 with respect to SEQ ID NO:1 or SEQ ID NO:61.

In a particular embodiment, the functional C-terminal truncated GDE polypeptide of the invention is deleted of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61; and is further deleted of:
at least one amino acid selected from amino acids at positions 1 to 428 with respect to SEQ ID NO:1 or SEQ ID NO:61, preferably at least 10, 15, 20, 30, 40, 50, 60, 80, 100, 150, 200, 250, 300, 350 or at least 400 consecutive amino acids selected from amino acids at positions 1 to 428 with respect to SEQ ID NO:1 or SEQ ID NO:61; and/or
at least one amino acid selected from amino acids at positions 668 to 769 with respect to SEQ ID NO:1 or SEQ ID NO:61, preferably at least 10, 15, 20, 30, 40, 50, 80 or at least 100 consecutive amino acids selected from amino acids at positions 668 to 769 with respect to SEQ ID NO:1 or SEQ ID NO:61; and/or
at least one amino acid selected from amino acids at positions 895 to 1087 with respect to SEQ ID NO:1 or SEQ ID NO:61, preferably at least 10, 15, 20, 30, 40, 50, 80, 100, 125, 150, 175, or at least 190 consecutive amino acids selected from amino acids at positions 895 to 1087 with respect to SEQ ID NO:1 or SEQ ID NO:61; and/or
at least one amino acid selected from amino acids at positions 1195 to 1232 with respect to SEQ ID NO:1 or SEQ ID NO:61, preferably at least 10, 15, 20 or at least 30 consecutive amino acids selected from amino acids at positions 1195 to 1232 with respect to SEQ ID NO:1 or SEQ ID NO:61.

In a further particular embodiment, the functional C-terminal truncated GDE polypeptide of the invention is deleted of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61; and is further deleted of:
at least one amino acid selected from amino acids at positions 1 to 428 with respect to SEQ ID NO:1 or SEQ ID NO:61, preferably at least 10, 15, 20, 30, 40, 50, 60, 80, 100, 110 or at least 120 consecutive amino acids selected from amino acids at positions 1 to 428 with respect to SEQ ID NO:1 or SEQ ID NO:61; and/or
at least one amino acid selected from amino acids at positions 668 to 769 with respect to SEQ ID NO:1 or SEQ ID NO:61, preferably at least 10, 15, 20, 30, 40, 50, 80 or at least 100 consecutive amino acids selected from amino acids at positions 668 to 769 with respect to SEQ ID NO:1 or SEQ ID NO:61; and/or
at least one amino acid selected from amino acids at positions 895 to 1087 with respect to SEQ ID NO:1 or SEQ ID NO:61, preferably at least 10, 15, 20, 30, 40, 50, 80, 100, 125, 150, 175, or at least 190 consecutive amino acids selected from amino acids at positions 895 to 1087 with respect to SEQ ID NO:1 or SEQ ID NO:61; and/or
at least one amino acid selected from amino acids at positions 1195 to 1232 with respect to SEQ ID NO:1 or SEQ ID NO:61, preferably at least 10, 15, 20 or at least 30 consecutive amino acids selected from amino acids at positions 1195 to 1232 with respect to SEQ ID NO:1 or SEQ ID NO:61.

In a particular embodiment, the functional C-terminal truncated GDE polypeptide of the invention is deleted of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61; and is further deleted of:
at least one amino acid selected from amino acids at positions 1 to 156 with respect to SEQ ID NO:1 or SEQ ID NO:61, preferably at least 15 consecutive amino acids, at least 50 consecutive amino acids or at least 100 consecutive amino acids selected from amino acids at positions 1 to 156 with respect to SEQ ID NO:1 or SEQ ID NO:61; and/or
at least one amino acid selected from amino acids at positions 361 to 428 with respect to SEQ ID NO:1 or SEQ ID NO:61, preferably at least 15 consecutive amino acids or at least 50 consecutive amino acids selected from amino acids at positions 361 to 428 with respect to SEQ ID NO:1 or SEQ ID NO:61; and/or
at least one amino acid selected from amino acids at positions 668 to 769 with respect to SEQ ID NO:1 or SEQ ID NO:61, preferably at least 15 consecutive amino acids, at least 50 consecutive amino acids or at least 100 consecutive amino acids selected from amino acids at positions 668 to 769 with respect to SEQ ID NO:1 or SEQ ID NO:61; and/or
at least one amino acid selected from amino acids at positions 895 to 1087 with respect to SEQ ID NO:1 or SEQ ID NO:61, preferably at least 15 consecutive amino acids, at least 50 consecutive amino acids, at least 100 consecutive amino acids, at least 150 consecutive amino acids, at least 175 consecutive amino acids or at least 190 consecutive amino acids selected from amino acids at positions 895 to 1087 with respect to SEQ ID NO:1 or SEQ ID NO:61; and/or
at least one amino acid selected from amino acids at positions 1195 to 1232 with respect to SEQ ID NO:1 or SEQ ID NO:61, preferably at least 15 consecutive amino acids selected from amino acids at positions 1195 to 1232 with respect to SEQ ID NO:1 or SEQ ID NO:61; and/or
at least one amino acid selected from amino acids at positions 223 to 320 with respect to SEQ ID NO:1 or SEQ ID NO:61, preferably at least 15 consecutive amino acids or at least 50 consecutive amino acids selected from amino acids at positions 223 to 320 with respect to SEQ ID NO:1 or SEQ ID NO:61; and/or
at least one amino acid selected from amino acids at positions 360 to 428 with respect to SEQ ID NO:1 or SEQ ID NO:61, preferably at least 15 consecutive amino acids or at least 50 consecutive amino acids selected from amino acids at positions 360 to 428 with respect to SEQ ID NO:1 or SEQ ID NO:61; and/or
at least one amino acid selected from amino acids at positions 669 to 720 with respect to SEQ ID NO:1 or SEQ ID NO:61, preferably at least 15 consecutive amino acids or at least 50 consecutive amino acids selected from amino acids at positions 669 to 720 with respect to SEQ ID NO:1 or SEQ ID NO:61; and/or
at least one amino acid selected from amino acids at positions 1 to 280 with respect to SEQ ID NO:1 or SEQ ID NO:61, preferably at least 15 consecutive amino acids, at least 50 consecutive amino acids or at least 100 consecutive amino acids selected from amino acids at positions 1 to 280 with respect to SEQ ID NO:1 or SEQ ID NO:61; and/or
at least one amino acid selected from amino acids at positions 1 to 425 with respect to SEQ ID NO:1 or SEQ ID NO:61, preferably at least 15 consecutive amino acids, at least 50 consecutive amino acids or at least 100 consecutive amino acids selected from amino acids at positions 1 to 425 with respect to SEQ ID NO:1 or SEQ ID NO:61; and/or
at least one amino acid selected from amino acids at positions 1 to 230 with respect to SEQ ID NO:1 or SEQ ID NO:61, preferably at least 15 consecutive amino acids, at least 50 consecutive amino acids or at least 100 consecutive amino acids selected from amino acids at positions 1 to 230 with respect to SEQ ID NO:1 or SEQ ID NO:61; and/or
at least one amino acid selected from amino acids at positions 1 to 15, with respect to SEQ ID NO:1 or SEQ ID NO:61, preferably at least 5 consecutive amino acids or at least 10 consecutive amino acids selected from amino acids at positions 1 to 15 with respect to SEQ ID NO:1 or SEQ ID NO:61; and/or
at least one amino acid selected from amino acids at positions 1 to 30 with respect to SEQ ID NO:1 or SEQ ID NO:61, preferably at least 15 consecutive amino acids or at least 25 consecutive amino acids selected from amino acids at positions 1 to 30 with respect to SEQ ID NO:1 or SEQ ID NO:61; and/or
at least one amino acid selected from amino acids at positions 1 to 81 with respect to SEQ ID NO:1 or SEQ ID NO:61, preferably at least 15 consecutive amino acids or at least 50 consecutive amino acids selected from amino acids at positions 1 to 81 with respect to SEQ ID NO:1 or SEQ ID NO:61; and/or
at least one amino acid selected from amino acids at positions 1 to 103 with respect to SEQ ID NO:1 or SEQ ID NO:61, preferably at least 15 consecutive amino acids or at least 50 consecutive amino acids selected from amino acids at positions 1 to 103 with respect to SEQ ID NO:1 or SEQ ID NO:61; and/or
at least one amino acid selected from amino acids at positions 1 to 129 with respect to SEQ ID NO:1 or SEQ ID NO:61, preferably at least 15 consecutive amino acids, at least 50 consecutive amino acids or at least 100 consecutive amino acids selected from amino acids at positions 1 to 129 with respect to SEQ ID NO:1 or SEQ ID NO:61.

In a further particular embodiment, the functional C-terminal truncated GDE polypeptide of the invention comprises a C-terminal deletion of amino acids from position 1421 to 1532 with respect to SEQ ID NO:1 or SEQ ID NO:61, in combination with at least one further deletion selected from the group consisting of:
deletion of amino acids from position 1 to 156 with respect to SEQ ID NO:1 or SEQ ID NO:61;
deletion of amino acids from position 361 to 428 with respect to SEQ ID NO:1 or SEQ ID NO:61;
deletion of amino acids from position 668 to 769 with respect to SEQ ID NO:1 or SEQ ID NO:61;
deletion of amino acids from position 895 to 1087 with respect to SEQ ID NO:1 or SEQ ID NO:61;
deletion of amino acids from position 1195 to 1232 with respect to SEQ ID NO:1 or SEQ ID NO:61;
deletion of amino acids from position 223 to 320 with respect to SEQ ID NO:1 or SEQ ID NO:61;
deletion of amino acids from position 360 to 428 with respect to SEQ ID NO:1 or SEQ ID NO:61;
deletion of amino acids from position 669 to 720 with respect to SEQ ID NO:1 or SEQ ID NO:61;
deletion of amino acids from position 1 to 280 with respect to SEQ ID NO:1 or SEQ ID NO:61;
deletion of amino acids from position 1 to 425 with respect to SEQ ID NO:1 or SEQ ID NO:61;
deletion of amino acids from position 1 to 230 with respect to SEQ ID NO:1 or SEQ ID NO:61;
deletion of amino acids from position 1 to 15 with respect to SEQ ID NO:1 or SEQ ID NO:61;
deletion of amino acids from position 1 to 30 with respect to SEQ ID NO:1 or SEQ ID NO:61;
deletion of amino acids from position 1 to 81 with respect to SEQ ID NO:1 or SEQ ID NO:61;
deletion of amino acids from position 1 to 103 with respect to SEQ ID NO:1 or SEQ ID NO:61; and
deletion of amino acids from position 1 to 129 with respect to SEQ ID NO:1 or SEQ ID NO:61.

In a further particular embodiment, the functional C-terminal truncated GDE polypeptide of the invention comprises a C-terminal deletion of amino acids from position 1421 to 1532 with respect to SEQ ID NO:1 or SEQ ID NO:61, in combination with one further deletion selected from the group consisting of:
deletion of amino acids from position 1 to 15 with respect to SEQ ID NO:1 or SEQ ID NO:61;
deletion of amino acids from position 1 to 30 with respect to SEQ ID NO:1 or SEQ ID NO:61;
deletion of amino acids from position 1 to 81 with respect to SEQ ID NO:1 or SEQ ID NO:61;
deletion of amino acids from position 1 to 103 with respect to SEQ ID NO:1 or SEQ ID NO:61; and
deletion of amino acids from position 1 to 129 with respect to SEQ ID NO:1 or SEQ ID NO:61.

For the sake of clarity, in this embodiment, the deletion relates to the deletion of all consecutive amino acids in the mentioned range of positions. For example, a functional C-terminal truncated GDE polypeptide comprising the deletion of amino acids from position 1 to 156 with respect to SEQ ID NO:1 corresponds to a GDE polypeptide which is deleted of all consecutive amino acids from position 1 to 156 with respect to SEQ ID NO:1.

Also for the sake of clarity, a functional C-terminal truncated GDE polypeptide comprising for example
the deletion of amino acids from position 1 to 156 with respect to SEQ ID NO:1; and
the deletion of amino acids from position 1 to 280 with respect to SEQ ID NO:1 corresponds to a GDE polypeptide which is deleted of all consecutive amino acids from position 1 to 280, since the range 1-156 is included in the range 1-280.

In addition, a functional C-terminal truncated GDE polypeptide comprising for example:
the deletion of amino acids from position 1 to 280 with respect to SEQ ID NO:1; and
the deletion of amino acids from position 223 to 320 with respect to SEQ ID NO:1 corresponds to a GDE polypeptide which is deleted of all consecutive amino acids from position 1 to 320, since the range 1-280 overlaps the range 223-320.

In another embodiment, the reference full-length human GDE sequence has an amino acid sequence as shown in SEQ ID NO:2 or SEQ ID NO:62, which correspond to GDE isoform 5.

The C-terminal truncated GDE polypeptide of the invention comprises a C-terminal deletion of at least one amino acid and of at most 112 amino acids selected from amino acids at positions 1404 to 1515 with respect to SEQ ID NO:2 or SEQ ID NO:62. In a particular embodiment, the C-terminal truncated GDE polypeptide comprises a C-terminal deletion of at least 1, 5, 10, 15, 20, 25, 30, 35, 40, 45, 50, 55, 60, 65, 70, 75, 80, 85, 90, 95, 100, 105, or at least 110 amino acids selected from amino acids at positions 1404 to 1515 with respect to SEQ ID NO:2 or SEQ ID NO:62.

In a particular embodiment, the C-terminal truncated GDE polypeptide comprises a C-terminal deletion of at least about 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62.

In a preferred embodiment, the C-terminal truncated GDE polypeptide comprises a C-terminal deletion of 112 consecutive amino acids from amino acids at positions 1404 to 1515 with respect to SEQ ID NO:2 or SEQ ID NO:62. For the sake of clarity, in this embodiment, the deletion relates to the deletion of all consecutive amino acids in the mentioned range of positions. This C-terminal deletion of 112 consecutive amino acids from amino acids at positions 1404 to 1515 with respect to SEQ ID NO:2 or SEQ ID NO:62 is hereafter called the deletion “ΔCter-2”.

In a particular embodiment, the functional C-terminal truncated GDE polypeptide of the invention which comprises a C-terminal deletion as described above, is further deleted of at least one amino acid with respect to SEQ ID NO:2 or SEQ ID NO:62, wherein the deleted amino acid(s) is at least one amino acid at positions 1-411, 651-752, 878-1070 and/or 1178-1215 with respect to SEQ ID NO:2 or SEQ ID NO:62. In a further particular embodiment, the functional C-terminal truncated GDE polypeptide of the invention which comprises a C-terminal deletion as described above is further deleted of at least about 10, 20, 30, 40, 50, 60, 75, 90, 100, 125, 150, 175, 190, 200, 225, 250, 275, 300, 325, 350, 375, 400, 425, 450, 475, 500 or at least about 525 amino acids, wherein the deleted amino acid(s) are selected from any amino acids at positions 1-411, 651-752, 878-1070, and/or 1178-1215 with respect to SEQ ID NO:2 or SEQ ID NO:62. In this embodiment, the deleted amino acids may be consecutive amino acids or non-consecutive amino acids, as long as they are selected from any amino acids at positions 1-411, 651-752, 878-1070 and/or 1178-1215 with respect to SEQ ID NO:2 or SEQ ID NO:62.

In a particular embodiment, the functional C-terminal truncated GDE polypeptide of the invention comprises at least the amino acid residues at positions 412-649, 753-875, 1071-1177, 1218-1515 with respect to SEQ ID NO:2 or SEQ ID NO:62.

In a particular embodiment, the functional C-terminal truncated GDE polypeptide of the invention comprises at least the amino acid residues at positions 412-650, 753-877, 1071-1177, 1216-1515 with respect to SEQ ID NO:2 or SEQ ID NO:62.

In a particular embodiment, the functional C-terminal truncated GDE polypeptide of the invention is deleted of at least one amino acid and of at most 112 amino acids selected from amino acids at positions 1404 to 1515 with respect to SEQ ID NO:2 or SEQ ID NO:62, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62; and is further deleted of:
at least one amino acid selected from amino acids at positions 1 to 411 with respect to SEQ ID NO:2 or SEQ ID NO:62, preferably at least 10, 15, 20, 30, 40, 50, 60, 80, 100, 150, 200, 250, 300, 350 or at least 400 consecutive amino acids selected from amino acids at positions 1 to 411 with respect to SEQ ID NO:2 or SEQ ID NO:62; and/or
at least one amino acid selected from amino acids at positions 651-752 with respect to SEQ ID NO:2 or SEQ ID NO:62, preferably at least 10, 15, 20, 30, 40, 50, 80 or at least 100 consecutive amino acids selected from amino acids at positions 651-752 with respect to SEQ ID NO:2 or SEQ ID NO:62; and/or
at least one amino acid selected from amino acids at positions 878-1070 with respect to SEQ ID NO:2 or SEQ ID NO:62, preferably at least 10, 15, 20, 30, 40, 50, 80, 100, 125, 150, 175, or at least 190 consecutive amino acids selected from amino acids at positions 878-1070 with respect to SEQ ID NO:2 or SEQ ID NO:62; and/or
at least one amino acid selected from amino acids at positions 1178-1215 with respect to SEQ ID NO:2 or SEQ ID NO:62, preferably at least 10, 15, 20 or at least 30 consecutive amino acids selected from amino acids at positions 1178-1215 with respect to SEQ ID NO:2 or SEQ ID NO:62.

In a particular embodiment, the functional C-terminal truncated GDE polypeptide of the invention is deleted of at least one amino acid and of at most 112 amino acids selected from amino acids at positions 1404 to 1515 with respect to SEQ ID NO:2 or SEQ ID NO:62, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62; and is further deleted of:
at least one amino acid selected from amino acids at positions 1 to 139 with respect to SEQ ID NO:2 or SEQ ID NO:62, preferably at least 15 consecutive amino acids, at least 50 consecutive amino acids or at least 100 consecutive amino acids selected from amino acids at positions 1 to 139 with respect to SEQ ID NO:2 or SEQ ID NO:62; and/or
at least one amino acid selected from amino acids at positions 344 to 411 with respect to SEQ ID NO:2 or SEQ ID NO:62, preferably at least 15 consecutive amino acids or at least 50 consecutive amino acids selected from amino acids at positions 344 to 411 with respect to SEQ ID NO:2 or SEQ ID NO:62; and/or
at least one amino acid selected from amino acids at positions 651 to 752 with respect to SEQ ID NO:2 or SEQ ID NO:62, preferably at least 15 consecutive amino acids, at least 50 consecutive amino acids or at least 100 consecutive amino acids selected from amino acids at positions 651 to 752 with respect to SEQ ID NO:2 or SEQ ID NO:62; and/or
at least one amino acid selected from amino acids at positions 878 to 1070 with respect to SEQ ID NO:2 or SEQ ID NO:62, preferably at least 15 consecutive amino acids, at least 50 consecutive amino acids, at least 100 consecutive amino acids, at least 150 consecutive amino acids, at least 175 consecutive amino acids or at least 190 consecutive amino acids selected from amino acids at positions 878 to 1070 with respect to SEQ ID NO:2 or SEQ ID NO:62; and/or
at least one amino acid selected from amino acids at positions 1178 to 1215 with respect to SEQ ID NO:2 or SEQ ID NO:62, preferably at least 15 consecutive amino acids selected from amino acids at positions 1178 to 1215 with respect to SEQ ID NO:2 or SEQ ID NO:62; and/or
at least one amino acid selected from amino acids at positions 206 to 303 with respect to SEQ ID NO:2 or SEQ ID NO:62, preferably at least 15 consecutive amino acids or at least 50 consecutive amino acids selected from amino acids at positions 206 to 303 with respect to SEQ ID NO:2 or SEQ ID NO:62; and/or
at least one amino acid selected from amino acids at positions 343 to 411 with respect to SEQ ID NO:2 or SEQ ID NO:62, preferably at least 15 consecutive amino acids or at least 50 consecutive amino acids selected from amino acids at positions 343 to 411 with respect to SEQ ID NO:2 or SEQ ID NO:62; and/or
at least one amino acid selected from amino acids at positions 652 to 703 with respect to SEQ ID NO:2 or SEQ ID NO:62, preferably at least 15 consecutive amino acids or at least 50 consecutive amino acids selected from amino acids at positions 652 to 703 with respect to SEQ ID NO:2 or SEQ ID NO:62; and/or
at least one amino acid selected from amino acids at positions 1 to 263 with respect to SEQ ID NO:2 or SEQ ID NO:62, preferably at least 15 consecutive amino acids, at least 50 consecutive amino acids or at least 100 consecutive amino acids selected from amino acids at positions 1 to 263 with respect to SEQ ID NO:2 or SEQ ID NO:62; and/or
at least one amino acid selected from amino acids at positions 1 to 408 with respect to SEQ ID NO:2 or SEQ ID NO:62, preferably at least 15 consecutive amino acids, at least 50 consecutive amino acids or at least 100 consecutive amino acids selected from amino acids at positions 1 to 408 with respect to SEQ ID NO:2 or SEQ ID NO:62; and/or
at least one amino acid selected from amino acids at positions 1 to 213 with respect to SEQ ID NO:2 or SEQ ID NO:62, preferably at least 15 consecutive amino acids, at least 50 consecutive amino acids or at least 100 consecutive amino acids selected from amino acids at positions 1 to 213 with respect to SEQ ID NO:2 or SEQ ID NO:62; and/or
at least one amino acid selected from amino acids at positions 1 to 13 with respect to SEQ ID NO:2 or SEQ ID NO:62, preferably at least 5 consecutive amino acids or at least 10 consecutive amino acids selected from amino acids at positions 1 to 13 with respect to SEQ ID NO:2 or SEQ ID NO:62; and/or
at least one amino acid selected from amino acids at positions 1 to 64 with respect to SEQ ID NO:2 or SEQ ID NO:62, preferably at least 15 consecutive amino acids or at least 50 consecutive amino acids selected from amino acids at positions 1 to 64 with respect to SEQ ID NO:2 or SEQ ID NO:62; and/or
at least one amino acid selected from amino acids at positions 1 to 86 with respect to SEQ ID NO:2 or SEQ ID NO:62, preferably at least 15 consecutive amino acids or at least 50 consecutive amino acids selected from amino acids at positions 1 to 86 with respect to SEQ ID NO:2 or SEQ ID NO:62; and/or
at least one amino acid selected from amino acids at positions 1 to 112 with respect to SEQ ID NO:2 or SEQ ID NO:62, preferably at least 15 consecutive amino acids, at least 50 consecutive amino acids or at least 100 consecutive amino acids selected from amino acids at positions 1 to 112 with respect to SEQ ID NO:2 or SEQ ID NO:62.

In a further particular embodiment, the functional C-terminal truncated GDE polypeptide of the invention comprises a C-terminal deletion of amino acids from position 1404 to 1515 with respect to SEQ ID NO:2 or SEQ ID NO:62, in combination with at least one further deletion selected from the group consisting of:
deletion of amino acids from position 1 to 139 with respect to SEQ ID NO:2 or SEQ ID NO:62;
deletion of amino acids from position 344 to 411 with respect to SEQ ID NO:2 or SEQ ID NO:62;
deletion of amino acids from position 651 to 752 with respect to SEQ ID NO:2 or SEQ ID NO:62;
deletion of amino acids from position 878 to 1070 with respect to SEQ ID NO:2 or SEQ ID NO:62;
deletion of amino acids from position 1178 to 1215 with respect to SEQ ID NO:2 or SEQ ID NO:62;
deletion of amino acids from position 206 to 303 with respect to SEQ ID NO:2 or SEQ ID NO:62;
deletion of amino acids from position 343 to 411 with respect to SEQ ID NO:2 or SEQ ID NO:62;
deletion of amino acids from position 652 to 703 with respect to SEQ ID NO:2 or SEQ ID NO:62;
deletion of amino acids from position 1 to 263 with respect to SEQ ID NO:2 or SEQ ID NO:62;
deletion of amino acids from position 1 to 408 with respect to SEQ ID NO:2 or SEQ ID NO:62;
deletion of amino acids from position 1 to 213 with respect to SEQ ID NO:2 or SEQ ID NO:62;
deletion of amino acids from position 1 to 13 with respect to SEQ ID NO:2 or SEQ ID NO:62;
deletion of amino acids from position 1 to 64 with respect to SEQ ID NO:2 or SEQ ID NO:62;
deletion of amino acids from position 1 to 86 with respect to SEQ ID NO:2 or SEQ ID NO:62; and
deletion of amino acids from position 1 to 112 with respect to SEQ ID NO:2 or SEQ ID NO:62.

In a further particular embodiment, the functional C-terminal truncated GDE polypeptide of the invention comprises a C-terminal deletion of amino acids from position 1404 to 1515 with respect to SEQ ID NO:2 or SEQ ID NO:62, in combination with one further deletion selected from the group consisting of:
deletion of amino acids from position 1 to 13 with respect to SEQ ID NO:2 or SEQ ID NO:62;
deletion of amino acids from position 1 to 64 with respect to SEQ ID NO:2 or SEQ ID NO:62;
deletion of amino acids from position 1 to 86 with respect to SEQ ID NO:2 or SEQ ID NO:62; and
deletion of amino acids from position 1 to 112 with respect to SEQ ID NO:2 or SEQ ID NO:62.

For the sake of clarity, in this embodiment, the deletion relates to the deletion of all consecutive amino acids in the mentioned range of positions. For example, a functional C-terminal truncated GDE polypeptide comprising the deletion of amino acids from position 1 to 139 with respect to SEQ ID NO:2 corresponds to a GDE polypeptide which is deleted of all consecutive amino acids from position 1 to 139 with respect to SEQ ID NO:2.

Also for the sake of clarity, a functional C-terminal truncated GDE polypeptide comprising for example
the deletion of amino acids from position 1 to 139 with respect to SEQ ID NO:2; and
the deletion of amino acids from position 1 to 263 with respect to SEQ ID NO:2 corresponds to a GDE polypeptide which is deleted of all consecutive amino acids from position 1 to 263, since the range 1-139 is included in the range 1-263.

In addition, a functional C-terminal truncated GDE polypeptide comprising for example:
the deletion of amino acids from position 1 to 263 with respect to SEQ ID NO:2; and
the deletion of amino acids from position 206 to 303 with respect to SEQ ID NO:2 corresponds to a GDE polypeptide which is deleted of all consecutive amino acids from position 1 to 303, since the range 1-263 overlaps the range 206-303.

In another embodiment, the reference full-length human GDE sequence has an amino acid sequence as shown in SEQ ID NO:3 or SEQ ID NO:63, which correspond to GDE isoform 6.

The C-terminal truncated GDE polypeptide of the invention comprises a C-terminal deletion of at least one amino acid and of at most 112 amino acids selected from amino acids at positions 1405 to 1516 with respect to SEQ ID NO:3 or SEQ ID NO:63. In a particular embodiment, the C-terminal truncated GDE polypeptide comprises a C-terminal deletion of at least 1, 5, 10, 15, 20, 25, 30, 35, 40, 45, 50, 55, 60, 65, 70, 75, 80, 85, 90, 95, 100, 105, or at least 110 amino acids selected from amino acids at positions 1405 to 1516 with respect to SEQ ID NO:3 or SEQ ID NO:63.

In a particular embodiment, the C-terminal truncated GDE polypeptide comprises a C-terminal deletion of at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63.

In a preferred embodiment, the C-terminal truncated GDE polypeptide comprises a C-terminal deletion of 112 consecutive amino acids from amino acids at positions 1405 to 1516 with respect to SEQ ID NO:3 or SEQ ID NO:63. For the sake of clarity, in this embodiment, the deletion relates to the deletion of all consecutive amino acids in the mentioned range of positions. This C-terminal deletion of 112 consecutive amino acids from amino acids at positions 1405 to 1516 with respect to SEQ ID NO:3 or SEQ ID NO:63 is hereafter called the deletion “ΔCter-3”.

In a particular embodiment, the functional C-terminal truncated GDE polypeptide of the invention which comprises a C-terminal deletion as described above, is further deleted of at least one amino acid with respect to SEQ ID NO:3 or SEQ ID NO:63, wherein the deleted amino acid(s) is at least one amino acid at positions 1-412, 652-753, 879-1071 and/or 1179-1216 with respect to SEQ ID NO:3 or SEQ ID NO:63. In a further particular embodiment, the functional C-terminal truncated GDE polypeptide of the invention which comprises a C-terminal deletion as described above is further deleted of at least about 10, 20, 30, 40, 50, 60, 75, 90, 100, 125, 150, 175, 190, 200, 225, 250, 275, 300, 325, 350, 375, 400, 425, 450, 475, 500 or at least about 525 amino acids, wherein the deleted amino acid(s) are selected from any amino acids at positions 1-412, 652-753, 879-1071, and/or 1179-1216 with respect to SEQ ID NO:3 or SEQ ID NO:63. In this embodiment, the deleted amino acids may be consecutive amino acids or non-consecutive amino acids, as long as they are selected from any amino acids at positions 1-412, 652-753, 879-1071 and/or 1179-1216 with respect to SEQ ID NO:3 or SEQ ID NO:63.

In a particular embodiment, the functional C-terminal truncated GDE polypeptide of the invention comprises at least the amino acid residues at positions 413-650, 754-876, 1072-1178, 1219-1516 with respect to SEQ ID NO:3 or SEQ ID NO:63.

In a particular embodiment, the functional C-terminal truncated GDE polypeptide of the invention comprises at least the amino acid residues at positions 413-651, 754-878, 1072-1178, 1217-1516 with respect to SEQ ID NO:3 or SEQ ID NO:63.

In a particular embodiment, the functional C-terminal truncated GDE polypeptide of the invention is deleted of at least one amino acid and of at most 112 amino acids selected from amino acids at positions 1405 to 1516 with respect to SEQ ID NO:3 or SEQ ID NO:63, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63; and is further deleted of:
at least one amino acid selected from amino acids at positions 1 to 412 with respect to SEQ ID NO:3 or SEQ ID NO:63, preferably at least 10, 15, 20, 30, 40, 50, 60, 80, 100, 150, 200, 250, 300, 350 or at least 400 consecutive amino acids selected from amino acids at positions 1 to 412 with respect to SEQ ID NO:3 or SEQ ID NO:63; and/or
at least one amino acid selected from amino acids at positions 652-753 with respect to SEQ ID NO:3 or SEQ ID NO:63, preferably at least 10, 15, 20, 30, 40, 50, 80 or at least 100 consecutive amino acids selected from amino acids at positions 652-753 with respect to SEQ ID NO:3 or SEQ ID NO:63; and/or
at least one amino acid selected from amino acids at positions 879-1071 with respect to SEQ ID NO:3 or SEQ ID NO:63, preferably at least 10, 15, 20, 30, 40, 50, 80, 100, 125, 150, 175, or at least 190 consecutive amino acids selected from amino acids at positions 879-1071 with respect to SEQ ID NO:3 or SEQ ID NO:63; and/or
at least one amino acid selected from amino acids at positions 1179-1216 with respect to SEQ ID NO:3 or SEQ ID NO:63, preferably at least 10, 15, 20 or at least 30 consecutive amino acids selected from amino acids at positions 1179-1216 with respect to SEQ ID NO:3 or SEQ ID NO:63.

In a particular embodiment, the functional C-terminal truncated GDE polypeptide of the invention is deleted of at least one amino acid and of at most 112 amino acids selected from amino acids at positions 1405 to 1516 with respect to SEQ ID NO:3 or SEQ ID NO:63, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63; and is further deleted of:
at least one amino acid selected from amino acids at positions 1 to 140 with respect to SEQ ID NO:3 or SEQ ID NO:63, preferably at least 15 consecutive amino acids, at least 50 consecutive amino acids or at least 100 consecutive amino acids selected from amino acids at positions 1 to 140 with respect to SEQ ID NO:3 or SEQ ID NO:63; and/or
at least one amino acid selected from amino acids at positions 345 to 412 with respect to SEQ ID NO:3 or SEQ ID NO:63, preferably at least 15 consecutive amino acids or at least 50 consecutive amino acids selected from amino acids at positions 345 to 412 with respect to SEQ ID NO:3 or SEQ ID NO:63; and/or
at least one amino acid selected from amino acids at positions 652 to 753 with respect to SEQ ID NO:3 or SEQ ID NO:63, preferably at least 15 consecutive amino acids, at least 50 consecutive amino acids or at least 100 consecutive amino acids selected from amino acids at positions 652 to 753 with respect to SEQ ID NO:3 or SEQ ID NO:63; and/or
at least one amino acid selected from amino acids at positions 879 to 1071 with respect to SEQ ID NO:3 or SEQ ID NO:63, preferably at least 15 consecutive amino acids, at least 50 consecutive amino acids, at least 100 consecutive amino acids, at least 150 consecutive amino acids, at least 175 consecutive amino acids or at least 190 consecutive amino acids selected from amino acids at positions 879 to 1071 with respect to SEQ ID NO:3 or SEQ ID NO:63; and/or
at least one amino acid selected from amino acids at positions 1179 to 1216 with respect to SEQ ID NO:3 or SEQ ID NO:63, preferably at least 15 consecutive amino acids selected from amino acids at positions 1179 to 1216 with respect to SEQ ID NO:3 or SEQ ID NO:63; and/or
at least one amino acid selected from amino acids at positions 207 to 304 with respect to SEQ ID NO:3 or SEQ ID NO:63, preferably at least 15 consecutive amino acids or at least 50 consecutive amino acids selected from amino acids at positions 207 to 304 with respect to SEQ ID NO:3 or SEQ ID NO:63; and/or
at least one amino acid selected from amino acids at positions 344 to 412 with respect to SEQ ID NO:3 or SEQ ID NO:63, preferably at least 15 consecutive amino acids or at least 50 consecutive amino acids selected from amino acids at positions 344 to 412 with respect to SEQ ID NO:3 or SEQ ID NO:63; and/or
at least one amino acid selected from amino acids at positions 653 to 704 with respect to SEQ ID NO:3 or SEQ ID NO:63, preferably at least 15 consecutive amino acids or at least 50 consecutive amino acids selected from amino acids at positions 653 to 704 with respect to SEQ ID NO:3 or SEQ ID NO:63; and/or
at least one amino acid selected from amino acids at positions 1 to 264 with respect to SEQ ID NO:3 or SEQ ID NO:63, preferably at least 15 consecutive amino acids, at least 50 consecutive amino acids or at least 100 consecutive amino acids selected from amino acids at positions 1 to 264 with respect to SEQ ID NO:3 or SEQ ID NO:63; and/or
at least one amino acid selected from amino acids at positions 1 to 409 with respect to SEQ ID NO:3 or SEQ ID NO:63, preferably at least 15 consecutive amino acids, at least 50 consecutive amino acids or at least 100 consecutive amino acids selected from amino acids at positions 1 to 409 with respect to SEQ ID NO:3 or SEQ ID NO:63;
at least one amino acid selected from amino acids at positions 1 to 214 with respect to SEQ ID NO:3 or SEQ ID NO:63, preferably at least 15 consecutive amino acids, at least 50 consecutive amino acids or at least 100 consecutive amino acids selected from amino acids at positions 1 to 214 with respect to SEQ ID NO:3 or SEQ ID NO:63; and/or
at least one amino acid selected from amino acids at positions 1 to 14 with respect to SEQ ID NO:3 or SEQ ID NO:63, preferably at least 5 consecutive amino acids or at least 10 consecutive amino acids selected from amino acids at positions 1 to 14 with respect to SEQ ID NO:3 or SEQ ID NO:63; and/or
at least one amino acid selected from amino acids at positions 1 to 65 with respect to SEQ ID NO:3 or SEQ ID NO:63, preferably at least 15 consecutive amino acids or at least 50 consecutive amino acids selected from amino acids at positions 1 to 65 with respect to SEQ ID NO:3 or SEQ ID NO:63; and/or
at least one amino acid selected from amino acids at positions 1 to 87 with respect to SEQ ID NO:3 or SEQ ID NO:63, preferably at least 15 consecutive amino acids or at least 50 consecutive amino acids selected from amino acids at positions 1 to 87 with respect to SEQ ID NO:3 or SEQ ID NO:63; and/or
at least one amino acid selected from amino acids at positions 1 to 113 with respect to SEQ ID NO:3 or SEQ ID NO:63, preferably at least 15 consecutive amino acids, at least 50 consecutive amino acids or at least 100 consecutive amino acids selected from amino acids at positions 1 to 113 with respect to SEQ ID NO:3 or SEQ ID NO:63.

In a further particular embodiment, the functional C-terminal truncated GDE polypeptide of the invention comprises a C-terminal deletion of amino acids from position 1405 to 1516 with respect to SEQ ID NO:3, in combination with at least one further deletion selected from the group consisting of:
deletion of amino acids from position 1 to 140 with respect to SEQ ID NO:3 or SEQ ID NO:63;
deletion of amino acids from position 345 to 412 with respect to SEQ ID NO:3 or SEQ ID NO:63;
deletion of amino acids from position 652 to 753 with respect to SEQ ID NO:3 or SEQ ID NO:63;
deletion of amino acids from position 879 to 1071 with respect to SEQ ID NO:3 or SEQ ID NO:63;
deletion of amino acids from position 1179 to 1216 with respect to SEQ ID NO:3 or SEQ ID NO:63;
deletion of amino acids from position 207 to 304 with respect to SEQ ID NO:3 or SEQ ID NO:63;
deletion of amino acids from position 344 to 412 with respect to SEQ ID NO:3 or SEQ ID NO:63;
deletion of amino acids from position 653 to 704 with respect to SEQ ID NO:3 or SEQ ID NO:63;
deletion of amino acids from position 1 to 264 with respect to SEQ ID NO:3 or SEQ ID NO:63;
deletion of amino acids from position 1 to 409 with respect to SEQ ID NO:3 or SEQ ID NO:63;
deletion of amino acids from position 1 to 214 with respect to SEQ ID NO:3 or SEQ ID NO:63;
deletion of amino acids from position 1 to 14 with respect to SEQ ID NO:3 or SEQ ID NO:63;
deletion of amino acids from position 1 to 65 with respect to SEQ ID NO:3 or SEQ ID NO:63;
deletion of amino acids from position 1 to 87 with respect to SEQ ID NO:3 or SEQ ID NO:63;
deletion of amino acids from position 1 to 113 with respect to SEQ ID NO:3 or SEQ ID NO:63.

In a further particular embodiment, the functional C-terminal truncated GDE polypeptide of the invention comprises a C-terminal deletion of amino acids from position 1405 to 1516 with respect to SEQ ID NO:3 or SEQ ID NO:63, in combination with one further deletion selected from the group consisting of:
deletion of amino acids from position 1 to 14 with respect to SEQ ID NO:3 or SEQ ID NO:63;
deletion of amino acids from position 1 to 65 with respect to SEQ ID NO:3 or SEQ ID NO:63;
deletion of amino acids from position 1 to 87 with respect to SEQ ID NO:3 or SEQ ID NO:63;
and
deletion of amino acids from position 1 to 113 with respect to SEQ ID NO:3 or SEQ ID NO:63.

For the sake of clarity, in this embodiment, the deletion relates to the deletion of all consecutive amino acids in the mentioned range of positions. For example, a functional C-terminal truncated GDE polypeptide comprising the deletion of amino acids from position 1 to 140 with respect to SEQ ID NO:3 corresponds to a GDE polypeptide which is deleted of all consecutive amino acids from position 1 to 140 with respect to SEQ ID NO:3.

Also for the sake of clarity, a functional C-terminal truncated GDE polypeptide comprising for example
the deletion of amino acids from position 1 to 140 with respect to SEQ ID NO:3; and
the deletion of amino acids from position 1 to 264 with respect to SEQ ID NO:3 corresponds to a GDE polypeptide which is deleted of all consecutive amino acids from position 1 to 264, since the range 1-140 is included in the range 1-264.

In addition, a functional C-terminal truncated GDE polypeptide comprising for example:
the deletion of amino acids from position 1 to 264 with respect to SEQ ID NO:3; and
the deletion of amino acids from position 207 to 304 with respect to SEQ ID NO:3 corresponds to a GDE polypeptide which is deleted of all consecutive amino acids from position 1 to 304, since the range 1-264 overlaps the range 207-304.

In a particular embodiment, the functional C-terminal truncated GDE polypeptide of the invention comprises a C-terminal deletion with respect to SEQ ID NO:1, SEQ ID NO:2, SEQ ID NO:3, SEQ ID NO:61, SEQ ID NO:62 or SEQ ID NO:63 wherein the C-terminal deletion is referred to as ΔCter-1, ΔCter-2 or ΔCter-3 in table 1:

TABLE 1

| Deletion | Position of the deleted amino acids |
|---|---|
| ΔCter-1 | Deletion of amino acids 1421-1532 with |
| | respect to SEQ ID NO: 1 or SEQ ID NO: 61 |
| ΔCter-2 | Deletion of amino acids 1404-1515 with |
| | respect to SEQ ID NO: 2 or SEQ ID NO: 62 |
| ΔCter-3 | Deletion of amino acids 1405-1516 with |
| | respect to SEQ ID NO: 3 or SEQ ID NO: 63 |

For the sake of clarity, Table 1 should be understood as follows. When the reference full-length GDE sequence is SEQ ID NO:1 or SEQ ID NO:61, the functional C-terminal truncated GDE polypeptide comprising a ΔCter-1 deletion corresponds to a functional C-terminal truncated GDE polypeptide derived from SEQ ID NO:1 or SEQ ID NO:61, which is deleted of all consecutive amino acids from position 1421 to 1532 with respect to SEQ ID NO:1 or SEQ ID NO:61. Accordingly, when the reference full-length GDE sequence is SEQ ID NO:2 or SEQ ID NO:62, the functional C-terminal truncated GDE polypeptide comprising a ΔCter-2 deletion corresponds to a functional truncated GDE polypeptide derived from SEQ ID NO:2 or SEQ ID NO:62, which is deleted of all consecutive amino acids from position 1404 to 1515 with respect to SEQ ID NO:2 or SEQ ID NO:62.

In a particular embodiment, the functional C-terminal truncated GDE polypeptide of the invention comprises, in addition to the C-terminal deletion such as a ΔCter-1, the ΔCter-2 or the ΔCter-3 deletion, a deletion or a combination of deletions with respect to SEQ ID NO:1, SEQ ID NO:2, SEQ ID NO:3, SEQ ID NO:61, SEQ ID NO:62 or SEQ ID NO:63 wherein the deletion(s) is(are) selected from any deletion referred to as Δ1, Δ2, Δ3, Δ4, Δ5, Δ6, Δ7, Δ8, Δ9, Δ10, Δ11, Δ12, and Δ13 in table 2:

TABLE 2

| | Position | Position | Position |
|---|---|---|---|
| | of the deleted | of the deleted | of the deleted |
| | amino acids with | amino acids with | amino acids with |
| | respect to | respect to | respect to |
| | SEQ ID NO: 1 or | SEQ ID NO: 2 or | SEQ ID NO: 3 or |
| Deletion | SEQ ID NO: 61 | SEQ ID NO: 62 | SEQ ID NO: 63 |
| Δ1 | 1-156 | 1-139 | 1-140 |
| Δ2 | 361-428 | 344-411 | 345-412 |
| Δ3 | 668-769 | 651-752 | 652-753 |
| Δ4 | 895-1087 | 878-1070 | 879-1071 |
| Δ5 | 223-320 | 206-303 | 207-304 |
| Δ6 | 360-428 | 343-411 | 344-412 |
| Δ7 | 669-720 | 652-703 | 653-704 |
| Δ8 | 1-280 | 1-263 | 1-264 |
| Δ9 | 1-15 | — | — |
| Δ10 | 1-30 | 1-13 | 1-14 |
| Δ11 | 1-81 | 1-64 | 1-65 |
| Δ12 | 1-103 | 1-86 | 1-87 |
| Δ13 | 1-129 | 1-112 | 1-113 |

In a particular embodiment, the functional truncated GDE polypeptide of the invention may comprise, in addition to the C-terminal deletion such as the ΔCter-1, ΔCter-2 or ΔCter-3 deletion, a combination of 2, 3, 4, 5, 6, 7, 8, 9, 10, 11, 12, or 13 deletions, wherein the deletion(s) is(are) selected from any deletion referred to as Δ1, Δ2, Δ3, Δ4, Δ5, Δ6, Δ7, Δ8, Δ9, Δ10, Δ11, Δ12, and Δ13 in table 2.

In a particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61; and
   (ii) one or more deletion(s) selected from any deletion referred to as Δ1, Δ2, Δ3, Δ4, Δ5, Δ6, Δ7, Δ8, Δ9, Δ10, Δ11, Δ12, and Δ13 in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:3 or SEQ ID NO:63.

In a further particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:3 or SEQ ID NO:63.

In a further particular embodiment:
the functional C-terminal truncated GDE polypeptide is derived from SEQ ID NO:1 or SEQ ID NO:61 and comprises:
   (i) the ΔCter-1 deletion as referred in table 1 with respect to SEQ ID NO:1 or SEQ ID NO:61; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) the ΔCter-2 deletion as referred in table 1 with respect to SEQ ID NO:2 or SEQ ID NO:62; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) the ΔCter-3 deletion as referred in table 1 with respect to SEQ ID NO:3 or SEQ ID NO:63; and
   (ii) one or more deletion(s) selected from any deletion referred to as Δ1, Δ2, Δ3, Δ4, Δ5, Δ6, Δ7, Δ8, Δ10, Δ11, Δ12, and Δ13 in table 2 with respect to SEQ ID NO:3 or SEQ ID NO:63.

In a further particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61; and
   (ii) a N-terminal deletion of at least one amino acid and of at most 132 amino acids selected from amino acids at positions 1 to 132 with respect to SEQ ID NO:1 or SEQ ID NO:61, in particular a N-terminal deletion of at least 15 consecutive amino acids, at least 25 consecutive amino acids, at least 30 consecutive amino acids, at least 50 consecutive amino acids, at least 75 consecutive amino acids, at least 80 consecutive amino acids, at least 81 consecutive amino acids, at least 100 consecutive amino acids, at least 103 consecutive amino acids, at least 125 consecutive amino acids, or at least 132 consecutive amino acids selected from amino acids at positions 1 to 132 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62; and
   (ii) a N-terminal deletion of at least one amino acid and of at most 115 amino acids selected from amino acids at positions 1 to 115 with respect to SEQ ID NO:2 or SEQ ID NO:62, in particular a N-terminal deletion of at least 10 consecutive amino acids, at least 13 consecutive amino acids, at least 15 consecutive amino acids, at least 25 consecutive amino acids, at least 50 consecutive amino acids, at least 60 consecutive amino acids, at least 64 consecutive amino acids, at least 75 consecutive amino acids, at least 80 consecutive amino acids, at least 85 consecutive amino acids, at least 86 consecutive amino acids, at least 100 consecutive amino acids, at least 110 consecutive amino acids, at least 112 consecutive amino acids, or at least 115 consecutive amino acids selected from amino acids at positions 1 to 115 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63; and
   (ii) a N-terminal deletion of at least one amino acid and of at most 116 amino acids selected from amino acids at positions 1 to 116 with respect to SEQ ID NO:3 or SEQ ID NO:63, in particular a N-terminal deletion of at least 10 consecutive amino acids, at least 14 consecutive amino acids, at least 15 consecutive amino acids, at least 25 consecutive amino acids, at least 50 consecutive amino acids, at least 60 consecutive amino acids, at least 65 consecutive amino acids, at least 75 consecutive amino acids, at least 80 consecutive amino acids, at least 85 consecutive amino acids, at least 87 consecutive amino acids, at least 100 consecutive amino acids, at least 110 consecutive amino acids, at least 113 consecutive amino acids, or at least 116 consecutive amino acids selected from amino acids at positions 1 to 116 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61; and
   (ii) a N-terminal deletion of at least one amino acid and of at most 132 amino acids selected from amino acids at positions 1 to 132 with respect to SEQ ID NO:1 or SEQ ID NO:61, in particular a N-terminal deletion of at least 15 consecutive amino acids, at least 25 consecutive amino acids, at least 30 consecutive amino acids, at least 50 consecutive amino acids, at least 75 consecutive amino acids, at least 80 consecutive amino acids, at least 81 consecutive amino acids, at least 100 consecutive amino acids, at least 103 consecutive amino acids, at least 125 consecutive amino acids, or at least 132 consecutive amino acids selected from amino acids at positions 1 to 132 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62; and
   (ii) a N-terminal deletion of at least one amino acid and of at most 115 amino acids selected from amino acids at positions 1 to 115 with respect to SEQ ID NO:2 or SEQ ID NO:62, in particular a N-terminal deletion of at least 10 consecutive amino acids, at least 13 consecutive amino acids, at least 15 consecutive amino acids, at least 25 consecutive amino acids, at least 50 consecutive amino acids, at least 60 consecutive amino acids, at least 64 consecutive amino acids, at least 75 consecutive amino acids, at least 80 consecutive amino acids, at least 85 consecutive amino acids, at least 86 consecutive amino acids, at least 100 consecutive amino acids, at least 110 consecutive amino acids, at least 112 consecutive amino acids, or at least 115 consecutive amino acids selected from amino acids at positions 1 to 115 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63; and
   (ii) a N-terminal deletion of at least one amino acid and of at most 116 amino acids selected from amino acids at positions 1 to 116 with respect to SEQ ID NO:3 or SEQ ID NO:63, in particular a N-terminal deletion of at least 10 consecutive amino acids, at least 14 consecutive amino acids, at least 15 consecutive amino acids, at least 25 consecutive amino acids, at least 50 consecutive amino acids, at least 60 consecutive amino acids, at least 65 consecutive amino acids, at least 75 consecutive amino acids, at least 80 consecutive amino acids, at least 85 consecutive amino acids, at least 87 consecutive amino acids, at least 100 consecutive amino acids, at least 110 consecutive amino acids, at least 113 consecutive amino acids, or at least 116 consecutive amino acids selected from amino acids at positions 1 to 116 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) the ΔCter-1 deletion as referred in table 1 with respect to SEQ ID NO:1 or SEQ ID NO:61; and
   (ii) a N-terminal deletion of at least one amino acid and of at most 132 amino acids selected from amino acids at positions 1 to 132 with respect to SEQ ID NO:1 or SEQ ID NO:61, in particular a N-terminal deletion of at least 15 consecutive amino acids, at least 25 consecutive amino acids, at least 30 consecutive amino acids, at least 50 consecutive amino acids, at least 75 consecutive amino acids, at least 80 consecutive amino acids, at least 81 consecutive amino acids, at least 100 consecutive amino acids, at least 103 consecutive amino acids, at least 125 consecutive amino acids, or at least 132 consecutive amino acids selected from amino acids at positions 1 to 132 with respect to SEQ ID NO:1 or SEQ ID NO:61;
      or
the functional C-terminal truncated GDE polypeptide is derived from SEQ ID NO:2 or SEQ ID NO:62 and comprises:
   (i) the ΔCter-2 deletion as referred in table 1 with respect to SEQ ID NO:2 or SEQ ID NO:62; and
   (ii) a N-terminal deletion of at least one amino acid and of at most 115 amino acids selected from amino acids at positions 1 to 115 with respect to SEQ ID NO:2 or SEQ ID NO:62, in particular a N-terminal deletion of at least 10 consecutive amino acids, at least 13 consecutive amino acids, at least 15 consecutive amino acids, at least 25 consecutive amino acids, at least 50 consecutive amino acids, at least 60 consecutive amino acids, at least 64 consecutive amino acids, at least 75 consecutive amino acids, at least 80 consecutive amino acids, at least 85 consecutive amino acids, at least 86 consecutive amino acids, at least 100 consecutive amino acids, at least 110 consecutive amino acids, at least 112 consecutive amino acids, or at least 115 consecutive amino acids selected from amino acids at positions 1 to 115 with respect to SEQ ID NO:2 or SEQ ID NO:62;
      or
the functional C-terminal truncated GDE polypeptide is derived from SEQ ID NO:3 or SEQ ID NO:63 and comprises:
   (i) the ΔCter-3 deletion as referred in table 1 with respect to SEQ ID NO:3 or SEQ ID NO:63; and
   (ii) a N-terminal deletion of at least one amino acid and of at most 116 amino acids selected from amino acids at positions 1 to 116 with respect to SEQ ID NO:3 or SEQ ID NO:63, in particular a N-terminal deletion of at least 10 consecutive amino acids, at least 14 consecutive amino acids, at least 15 consecutive amino acids, at least 25 consecutive amino acids, at least 50 consecutive amino acids, at least 60 consecutive amino acids, at least 65 consecutive amino acids, at least 75 consecutive amino acids, at least 80 consecutive amino acids, at least 85 consecutive amino acids, at least 87 consecutive amino acids, at least 100 consecutive amino acids, at least 110 consecutive amino acids, at least 113 consecutive amino acids, or at least 116 consecutive amino acids selected from amino acids at positions 1 to 116 with respect to SEQ ID NO:3 or SEQ ID NO:63.

In a particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61; and
   (ii) a deletion or a combination of deletions selected from the Δ9, Δ10, Δ11, Δ12 and Δ13 deletions as referred in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62; and
   (ii) a deletion or a combination of deletions selected from the Δ10, Δ11, Δ12 and Δ13 deletions as referred in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63; and
   (ii) a deletion or a combination of deletions selected from the Δ10, Δ11, Δ12 and Δ13 deletions as referred in table 2 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61; and
   (ii) a deletion or a combination of deletions selected from the Δ9, Δ10, Δ11, Δ12 and Δ13 deletions as referred in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62; and
   (ii) a deletion or a combination of deletions selected from the Δ10, Δ11, Δ12 and Δ13 deletions as referred in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63; and
   (ii) a deletion or a combination of deletions selected from the Δ10, Δ11, Δ12 and Δ13 deletions as referred in table 2 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) the ΔCter-1 deletion as referred in table 1 with respect to SEQ ID NO:1 or SEQ ID NO:61; and
   (ii) a deletion or a combination of deletions selected from the Δ9, Δ10, Δ11, Δ12 and Δ13 deletions as referred in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) the ΔCter-2 deletion as referred in table 1 with respect to SEQ ID NO:2 or SEQ ID NO:62; and
   (ii) a deletion or a combination of deletions selected from the Δ10, Δ11, Δ12 and Δ13 deletions as referred in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) the ΔCter-3 deletion as referred in table 1 with respect to SEQ ID NO:3 or SEQ ID NO:63; and
   (ii) a deletion or a combination of deletions selected from the Δ10, Δ11, Δ12 and Δ13 deletions as referred in table 2 with respect to SEQ ID NO:3 or SEQ ID NO:63.

In a particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61;
   (ii) a deletion or a combination of deletions as referred in table 2 with respect to SEQ ID NO:1 or SEQ ID NO:61 and
   (iii) a N-terminal deletion of at least one amino acid and of at most 132 amino acids selected from amino acids at positions 1 to 132 with respect to SEQ ID NO:1 or SEQ ID NO:61, in particular a N-terminal deletion of at least 15 consecutive amino acids, at least 25 consecutive amino acids, at least 30 consecutive amino acids, at least 50 consecutive amino acids, at least 75 consecutive amino acids, at least 80 consecutive amino acids, at least 81 consecutive amino acids, at least 100 consecutive amino acids, at least 103 consecutive amino acids, at least 125 consecutive amino acids, or at least 132 consecutive amino acids selected from amino acids at positions 1 to 132 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62;
   (ii) a deletion or a combination of deletions as referred in table 2 with respect to SEQ ID NO:2 or SEQ ID NO:62; and
   (iii) a N-terminal deletion of at least one amino acid and of at most 115 amino acids selected from amino acids at positions 1 to 115 with respect to SEQ ID NO:2 or SEQ ID NO:62, in particular a N-terminal deletion of at least 10 consecutive amino acids, at least 13 consecutive amino acids, at least 15 consecutive amino acids, at least 25 consecutive amino acids, at least 50 consecutive amino acids, at least 60 consecutive amino acids, at least 64 consecutive amino acids, at least 75 consecutive amino acids, at least 80 consecutive amino acids, at least 85 consecutive amino acids, at least 86 consecutive amino acids, at least 100 consecutive amino acids, at least 110 consecutive amino acids, at least 112 consecutive amino acids, or at least 115 consecutive amino acids selected from amino acids at positions 1 to 115 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63;
   (ii) a deletion or a combination of deletions as referred in table 2 with respect to SEQ ID NO:3 or SEQ ID NO:63; and
   (iii) a N-terminal deletion of at least one amino acid and of at most 116 amino acids selected from amino acids at positions 1 to 116 with respect to SEQ ID NO:3 or SEQ ID NO:63, in particular a N-terminal deletion of at least 10 consecutive amino acids, at least 14 consecutive amino acids, at least 15 consecutive amino acids, at least 25 consecutive amino acids, at least 50 consecutive amino acids, at least 60 consecutive amino acids, at least 65 consecutive amino acids, at least 75 consecutive amino acids, at least 80 consecutive amino acids, at least 85 consecutive amino acids, at least 87 consecutive amino acids, at least 100 consecutive amino acids, at least 110 consecutive amino acids, at least 113 consecutive amino acids, or at least 116 consecutive amino acids selected from amino acids at positions 1 to 116 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61;
   (ii) a deletion or a combination of deletions as referred in table 2 with respect to SEQ ID NO:1 or SEQ ID NO:61 and
   (iii) a N-terminal deletion of at least one amino acid and of at most 132 amino acids selected from amino acids at positions 1 to 132 with respect to SEQ ID NO:1 or SEQ ID NO:61, in particular a N-terminal deletion of at least 15 consecutive amino acids, at least 25 consecutive amino acids, at least 30 consecutive amino acids, at least 50 consecutive amino acids, at least 75 consecutive amino acids, at least 80 consecutive amino acids, at least 81 consecutive amino acids, at least 100 consecutive amino acids, at least 103 consecutive amino acids, at least 125 consecutive amino acids, or at least 132 consecutive amino acids selected from amino acids at positions 1 to 132 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62;
   (ii) a deletion or a combination of deletions as referred in table 2 with respect to SEQ ID NO:2 or SEQ ID NO:62; and
   (iii) a N-terminal deletion of at least one amino acid and of at most 115 amino acids selected from amino acids at positions 1 to 115 with respect to SEQ ID NO:2 or SEQ ID NO:62, in particular a N-terminal deletion of at least 10 consecutive amino acids, at least 13 consecutive amino acids, at least 15 consecutive amino acids, at least 25 consecutive amino acids, at least 50 consecutive amino acids, at least 60 consecutive amino acids, at least 64 consecutive amino acids, at least 75 consecutive amino acids, at least 80 consecutive amino acids, at least 85 consecutive amino acids, at least 86 consecutive amino acids, at least 100 consecutive amino acids, at least 110 consecutive amino acids, at least 112 consecutive amino acids, or at least 115 consecutive amino acids selected from amino acids at positions 1 to 115 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63;
   (ii) a deletion or a combination of deletions as referred in table 2 with respect to SEQ ID NO:3 or SEQ ID NO:63; and
   (iii) a N-terminal deletion of at least one amino acid and of at most 116 amino acids selected from amino acids at positions 1 to 116 with respect to SEQ ID NO:3 or SEQ ID NO:63, in particular a N-terminal deletion of at least 10 consecutive amino acids, at least 14 consecutive amino acids, at least 15 consecutive amino acids, at least 25 consecutive amino acids, at least 50 consecutive amino acids, at least 60 consecutive amino acids, at least 65 consecutive amino acids, at least 75 consecutive amino acids, at least 80 consecutive amino acids, at least 85 consecutive amino acids, at least 87 consecutive amino acids, at least 100 consecutive amino acids, at least 110 consecutive amino acids, at least 113 consecutive amino acids, or at least 116 consecutive amino acids selected from amino acids at positions 1 to 116 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) the ΔCter-1 deletion as referred in table 1 with respect to SEQ ID NO:1 or SEQ ID NO:61; and
   (ii) a deletion or a combination of deletions as referred in table 2 with respect to SEQ ID NO:1 or SEQ ID NO:61 and
   (iii) a N-terminal deletion of at least one amino acid and of at most 132 amino acids selected from amino acids at positions 1 to 132 with respect to SEQ ID NO:1 or SEQ ID NO:61, in particular a N-terminal deletion of at least 15 consecutive amino acids, at least 25 consecutive amino acids, at least 30 consecutive amino acids, at least 50 consecutive amino acids, at least 75 consecutive amino acids, at least 80 consecutive amino acids, at least 81 consecutive amino acids, at least 100 consecutive amino acids, at least 103 consecutive amino acids, at least 125 consecutive amino acids, or at least 132 consecutive amino acids selected from amino acids at positions 1 to 132 with respect to SEQ ID NO:1 or SEQ ID NO:61;
      or
the functional C-terminal truncated GDE polypeptide is derived from SEQ ID NO:2 or SEQ ID NO:62 and comprises:
   (i) the ΔCter-2 deletion as referred in table 1 with respect to SEQ ID NO:2 or SEQ ID NO:62; (ii) a deletion or a combination of deletions as referred in table 2 with respect to SEQ ID NO:2 or SEQ ID NO:62; and
   (iii) a N-terminal deletion of at least one amino acid and of at most 115 amino acids selected from amino acids at positions 1 to 115 with respect to SEQ ID NO:2 or SEQ ID NO:62, in particular a N-terminal deletion of at least 10 consecutive amino acids, at least 13 consecutive amino acids, at least 15 consecutive amino acids, at least 25 consecutive amino acids, at least 50 consecutive amino acids, at least 60 consecutive amino acids, at least 64 consecutive amino acids, at least 75 consecutive amino acids, at least 80 consecutive amino acids, at least 85 consecutive amino acids, at least 86 consecutive amino acids, at least 100 consecutive amino acids, at least 110 consecutive amino acids, at least 112 consecutive amino acids, or at least 115 consecutive amino acids selected from amino acids at positions 1 to 115 with respect to SEQ ID NO:2 or SEQ ID NO:62;
      or
the functional C-terminal truncated GDE polypeptide is derived from SEQ ID NO:3 or SEQ ID NO:63 and comprises:
   (i) the ΔCter-3 deletion as referred in table 1 with respect to SEQ ID NO:3 or SEQ ID NO:63;
   (ii) a deletion or a combination of deletions as referred in table 2 with respect to SEQ ID NO:3 or SEQ ID NO:63; and
   (iii) a N-terminal deletion of at least one amino acid and of at most 116 amino acids selected from amino acids at positions 1 to 116 with respect to SEQ ID NO:3 or SEQ ID NO:63, in particular a N-terminal deletion of at least 10 consecutive amino acids, at least 14 consecutive amino acids, at least 15 consecutive amino acids, at least 25 consecutive amino acids, at least 50 consecutive amino acids, at least 60 consecutive amino acids, at least 65 consecutive amino acids, at least 75 consecutive amino acids, at least 80 consecutive amino acids, at least 85 consecutive amino acids, at least 87 consecutive amino acids, at least 100 consecutive amino acids, at least 110 consecutive amino acids, at least 113 consecutive amino acids, or at least 116 consecutive amino acids selected from amino acids at positions 1 to 116 with respect to SEQ ID NO:3 or SEQ ID NO:63.

In a particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61;
   (ii) a deletion or a combination of deletions as referred in table 2 with respect to SEQ ID NO:1 or SEQ ID NO:61 and
   (iii) a deletion or a combination of deletions selected from the Δ9, Δ10, Δ11, Δ12 and Δ13 deletions as referred in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62;
   (ii) a deletion or a combination of deletions as referred in table 2 with respect to SEQ ID NO:2 or SEQ ID NO:62; and
   (iii) a deletion or a combination of deletions selected from the Δ10, Δ11, Δ12 and Δ13 deletions as referred in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63;
   (ii) a deletion or a combination of deletions as referred in table 2 with respect to SEQ ID NO:3 or SEQ ID NO:63; and
   (iii) a deletion or a combination of deletions selected from the Δ10, Δ11, Δ12 and Δ13 deletions as referred in table 2 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) a C-terminal deletion of at least one amino acid and of at most 112 amino acids selected from amino acids at positions 1421 to 1532 with respect to SEQ ID NO:1 or SEQ ID NO:61, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1421 to 1532 with respect to SEQ ID NO:1 or SEQ ID NO:61;
   (ii) a deletion or a combination of deletions as referred in table 2 with respect to SEQ ID NO:1 or SEQ ID NO:61 and
   (iii) a deletion or a combination of deletions selected from the Δ9, Δ10, Δ11, Δ12 and Δ13 deletions as referred in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) a C-terminal deletion of at least one amino acid and of at most 112 amino acids selected from amino acids at positions 1404 to 1515 with respect to SEQ ID NO:2 or SEQ ID NO:62, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1404 to 1515 with respect to SEQ ID NO:2 or SEQ ID NO:62;
   (ii) a deletion or a combination of deletions as referred in table 2 with respect to SEQ ID NO:2 or SEQ ID NO:62; and
   (iii) a deletion or a combination of deletions selected from the Δ10, Δ11, Δ12 and Δ13 deletions as referred in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) a C-terminal deletion of at least one amino acid and of at most 112 amino acids selected from amino acids at positions 1405 to 1516 with respect to SEQ ID NO:3 or SEQ ID NO:63, preferably at least 5, 10, 15, 20, 25, 30, 35, 40, 45, 50, 55, 60, 65, 70, 75, 80, 85, 90, 95, 100, 105, or at least 110 consecutive amino acids selected from amino acids at positions 1405 to 1516 with respect to SEQ ID NO:3 or SEQ ID NO:63;
   (ii) a deletion or a combination of deletions as referred in table 2 with respect to SEQ ID NO:3 or SEQ ID NO:63; and
   (iii) a deletion or a combination of deletions selected from the Δ10, Δ11, Δ12 and Δ13 deletions as referred in table 2 with respect to SEQ ID NO:3 or SEQ ID NO:63.

In another particular embodiment:
the functional C-terminal truncated GDE polypeptide is derived from SEQ ID NO:1 or SEQ ID NO:61 and comprises:
   (i) the ΔCter-1 deletion as referred in table 1 with respect to SEQ ID NO:1 or SEQ ID NO:61;
   (ii) a deletion or a combination of deletions as referred in table 2 with respect to SEQ ID NO:1 or SEQ ID NO:61 and
   (iii) a deletion or a combination of deletions selected from the Δ9, Δ10, Δ11, Δ12 and Δ13 deletions as referred in table 2 with respect to SEQ ID NO:1 or SEQ ID NO:61;
      or
the functional C-terminal truncated GDE polypeptide is derived from SEQ ID NO:2 or SEQ ID NO:62 and comprises:
   (i) the ΔCter-2 deletion as referred in table 1 with respect to SEQ ID NO:2 or SEQ ID NO:62;
   (ii) a deletion or a combination of deletions as referred in table 2 with respect to SEQ ID NO:2 or SEQ ID NO:62; and
   (iii) a deletion or a combination of deletions selected from the Δ10, Δ11, Δ12 and Δ13 deletions as referred in table 2 with respect to SEQ ID NO:2 or SEQ ID NO:62;
      or
the functional C-terminal truncated GDE polypeptide is derived from SEQ ID NO:3 or SEQ ID NO:63 and comprises:
   (i) the ΔCter-3 deletion as referred in table 1 with respect to SEQ ID NO:3 or SEQ ID NO:63;
   (ii) a deletion or a combination of deletions as referred in table 2 with respect to SEQ ID NO:3 or SEQ ID NO:63; and (iii) a deletion or a combination of deletions selected from the Δ10, Δ11, Δ12 and Δ13 deletions as referred in table 2 with respect to SEQ ID NO:3 or SEQ ID NO:63.

In a further particular embodiment, the functional C-terminal truncated GDE polypeptide according to the invention comprises or consists of the sequence SEQ ID NO:7 or SEQ ID NO:64, in particular SEQ ID NO:7. The functional C-terminal truncated GDE polypeptide may comprise one or more amino acid modifications such as amino acid insertion, deletion and/or substitution, as compared to SEQ ID NO:7 or SEQ ID NO:64, in particular SEQ ID NO:7. In particular, the functional C-terminal truncated GDE polypeptide may comprise 1, 2, 3, 4 or 5 amino acid modifications as compared to SEQ ID NO:7 or SEQ ID NO:64, in particular SEQ ID NO:7. In particular, the functional C-terminal truncated GDE polypeptide may have at least 80, 81, 82, 83, 84, 85, 86, 87, 88, 89, 90, 91, 92, 93, 94, 95, 96, 97, 98 or at least 99 percent sequence identity to SEQ ID NO:7 or SEQ ID NO:64, in particular SEQ ID NO:7.

In a further particular embodiment, the functional C-terminal truncated GDE polypeptide according to the invention comprises or consists of the sequence SEQ ID NO:8 or SEQ ID NO:65, in particular SEQ ID NO:8. The functional C-terminal truncated GDE polypeptide may comprise one or more amino acid modifications such as amino acid insertion, deletion and/or substitution, as compared to SEQ ID NO:8 or SEQ ID NO:65, in particular SEQ ID NO:8. In particular, the functional C-terminal truncated GDE polypeptide may comprise 1, 2, 3, 4 or 5 amino acid modifications as compared to SEQ ID NO:8 or SEQ ID NO:65, in particular SEQ ID NO:8. In particular, the functional C-terminal truncated GDE polypeptide may have at least 80, 81, 82, 83, 84, 85, 86, 87, 88, 89, 90, 91, 92, 93, 94, 95, 96, 97, 98 or at least 99 percent sequence identity to SEQ ID NO:8 or SEQ ID NO:65, in particular SEQ ID NO:8.

In a further particular embodiment, the functional C-terminal truncated GDE polypeptide according to the invention comprises or consists of the sequence SEQ ID NO:9 or SEQ ID NO:66, in particular SEQ ID NO:9. The functional C-terminal truncated GDE polypeptide may comprise one or more amino acid modifications such as amino acid insertion, deletion and/or substitution, as compared to SEQ ID NO:9 or SEQ ID NO:66, in particular SEQ ID NO:9. In particular, the functional C-terminal truncated GDE polypeptide may comprise 1, 2, 3, 4 or 5 amino acid modifications as compared to SEQ ID NO:9 or SEQ ID NO:66, in particular SEQ ID NO:9. In particular, the functional C-terminal truncated GDE polypeptide may have at least 80, 81, 82, 83, 84, 85, 86, 87, 88, 89, 90, 91, 92, 93, 94, 95, 96, 97, 98 or at least 99 percent sequence identity to SEQ ID NO:9 or SEQ ID NO:66, in particular SEQ ID NO:9.

In another particular embodiment, the functional truncated GDE polypeptide of the invention is selected from the group consisting of:
SEQ ID NO:25: a functional C-terminal truncated GDE polypeptide comprising a C-terminal deletion of amino acids from position 1421 to 1532; and a deletion of amino acids from position 1 to 156 with respect to SEQ ID NO:1;
SEQ ID NO:26: a functional C-terminal truncated GDE polypeptide comprising a C-terminal deletion of amino acids from position 1421 to 1532; and a deletion of amino acids from position 361 to 428 with respect to SEQ ID NO:1;
SEQ ID NO:27: a functional C-terminal truncated GDE polypeptide comprising a C-terminal deletion of amino acids from position 1421 to 1532; and a deletion of amino acids from position 668 to 769 with respect to SEQ ID NO:1;
SEQ ID NO:28: a functional C-terminal truncated GDE polypeptide comprising a C-terminal deletion of amino acids from position 1421 to 1532; a deletion of amino acids from position 361 to 428; and a deletion of amino acids from position 668 to 769 with respect to SEQ ID NO:1;
SEQ ID NO:29: a functional C-terminal truncated GDE polypeptide comprising a C-terminal deletion of amino acids from position 1421 to 1532; and a deletion of amino acids from position 895 to 1087 with respect to SEQ ID NO:1;
SEQ ID NO:30: a functional C-terminal truncated GDE polypeptide comprising a C-terminal deletion of amino acids from position 1421 to 1532; a deletion of amino acids from position 223 to 320; a deletion of amino acids from position 360 to 428; and a deletion of amino acids from position 669 to 720 with respect to SEQ ID NO:1;
SEQ ID NO:31: a functional C-terminal truncated GDE polypeptide comprising a C-terminal deletion of amino acids from position 1421 to 1532; and a deletion of amino acids from position 1 to 280 with respect to SEQ ID NO:1;
SEQ ID NO:32 a functional C-terminal truncated GDE polypeptide comprising a C-terminal deletion of amino acids from position 1421 to 1532; and a deletion of amino acids from position 1 to 425 with respect to SEQ ID NO:1;
SEQ ID NO:33: a functional C-terminal truncated GDE polypeptide comprising a C-terminal deletion of amino acids from position 1421 to 1532; and a deletion of amino acids from position 1 to 230 with respect to SEQ ID NO:1
SEQ ID NO:10: a functional C-terminal truncated GDE polypeptide comprising a C-terminal deletion of amino acids from position 1421 to 1532 and a deletion of amino acids from position 1-15 with respect to SEQ ID NO:1;
SEQ ID NO:11: a functional C-terminal truncated GDE polypeptide comprising a C-terminal deletion of amino acids from position 1421 to 1532 and a deletion of amino acids from position 1-30 with respect to SEQ ID NO:1;
SEQ ID NO:12: a functional C-terminal truncated GDE polypeptide comprising a C-terminal deletion of amino acids from position 1421 to 1532 and a deletion of amino acids from position 1-81 with respect to SEQ ID NO:1;
SEQ ID NO:13: a functional C-terminal truncated GDE polypeptide comprising a C-terminal deletion of amino acids from position 1421 to 1532 and a deletion of amino acids from position 1-103 with respect to SEQ ID NO:1; and
SEQ ID NO:14: a functional C-terminal truncated GDE polypeptide comprising a C-terminal deletion of amino acids from position 1421 to 1532 and a deletion of amino acids from position 1-129 with respect to SEQ ID NO:1.

When the deletion is a N-terminal deletion, a methionine may be added at the N-terminal end of the sequence. For example, SEQ ID NO:32 comprises a deletion of amino acids from position 1 to 425 with respect to SEQ ID NO:1 and an addition of a methionine at the N-terminal end of the sequence resulting from this deletion.

In a further particular embodiment, the functional truncated GDE polypeptide of the invention comprises or consists of a sequence selected from SEQ ID NO:25 to 33 and SEQ ID NO:10 to 14, in particular a sequence selected from SEQ ID NO:10 to 14, in particular a sequence selected from SEQ ID NO:10, SEQ ID NO:11, SEQ ID NO:12, SEQ ID NO:13 and SEQ ID NO:14. The functional truncated GDE polypeptide may comprise one or more amino acid modifications such as amino acid insertion, deletion and/or substitution, as compared to SEQ ID NO:25 to 33 and SEQ ID NO:10 to 14, in particular a sequence selected from SEQ ID NO: 10 to 14, in particular a sequence selected from SEQ ID NO:10, SEQ ID NO:11, SEQ ID NO:12, SEQ ID NO:13 and SEQ ID NO:14. In particular, the functional truncated GDE polypeptide may comprise 1, 2, 3, 4 or 5 amino acid modifications as compared to SEQ ID NO:25 to 33 and SEQ ID NO:10 to 14, in particular a sequence selected from SEQ ID NO: 10 to 14, in particular a sequence selected from SEQ ID NO:10, SEQ ID NO:11, SEQ ID NO:12, SEQ ID NO:13 and SEQ ID NO:14. In particular, the functional truncated GDE polypeptide may have at least 80, 85, 90, 95, 96, 97, 98 or at least 99 percent sequence identity to SEQ ID NO:25 to 33 and SEQ ID NO:10 to 14, in particular a sequence selected from SEQ ID NO: 10 to 14, in particular a sequence selected from SEQ ID NO:10, SEQ ID NO:11, SEQ ID NO:12, SEQ ID NO:13 and SEQ ID NO:14.

2—Nucleic Acid Molecule Encoding the C-Terminal Truncated GDE Polypeptide

In another aspect, the invention relates to a nucleic acid molecule encoding the functional C-terminal truncated GDE polypeptide as defined above.

The term “nucleic acid molecule” (or nucleic acid sequence) refers to a DNA or RNA molecule in single or double stranded form, particularly a DNA encoding a functional C-terminal truncated GDE polypeptide according to the invention.

According to the present invention, the nucleic acid molecule encoding the functional C-terminal truncated GDE polypeptide is small enough to be packaged into a gene therapy vector.

By “gene therapy vector” is meant any vector suitable for gene therapy. In particular, the gene therapy vector may be a plasmid or a recombinant virus such as a viral vector derived from a retrovirus or a lentivirus. Preferably, the viral vector is an AAV vector, such as an AAV vector suitable for transducing liver tissues or muscle cells. Extensive experience in clinical trials and in preclinical model of muscle diseases indicates adeno-associated virus (AAV) as the vector of choice for in vivo gene therapy for GSDIII. These vectors efficiently transduce liver and muscle, their production is scalable and compared to other gene therapy vectors they have a relatively low immunogenicity profile. However, one of the biggest limitations in the use of AAV for gene replacement is their limited encapsidation size limit (about 5 kb). Indeed, during recombinant AAV production, genomes larger than 5 kb are encapsidated with low efficacy and the resulting AAV may contain fragmented genomes reducing the efficacy of gene transfer.

In a preferred embodiment, the nucleic acid molecule encoding the functional C-terminal truncated GDE polypeptide is small enough to be packaged into an AAV vector. Preferably, the size of the nucleic acid molecule encoding the functional C-terminal truncated GDE polypeptide is less than about 4.5 kb, such as less than 4.4, 4.3, 4.2, 4.1, 4, 3.9, 3.8, 3.7, 3.6, 3.5, 3.4, 3.3, 3.2, 3.1, or 3 kb. Preferably, the nucleic acid molecule encoding the functional C-terminal truncated GDE polypeptide is less than about 4.4 kb, such as less than 4.3, 4.2 or 4.1 kb.

In a particular embodiment, the size of the nucleic acid molecule encoding the functional C-terminal truncated GDE polypeptide is of at least 1.8 kb, such as at least 1.9, 2, 2.1 or 2.2 kb. In a particular embodiment, the size of the nucleic acid molecule encoding the functional C-terminal truncated GDE polypeptide is of at least 1.8 kb and at most 4.5 kb. Preferably, the size of the nucleic acid molecule encoding the functional C-terminal truncated GDE polypeptide is of at least 2 kb and at most 4.4 kb. In a particular embodiment, the size of the nucleic acid molecule encoding the functional C-terminal truncated GDE polypeptide is of at least 2.2 kb and at most 4.3 kb.

The sequence of the nucleic acid molecule of the invention, encoding the functional C-terminal truncated GDE polypeptide may be optimized for expression of the GDE polypeptide in vivo. Sequence optimization may include a number of changes in a nucleic acid sequence, including codon optimization, increase of GC content, decrease of the number of CpG islands, decrease of the number of alternative open reading frames (ARFs) and decrease of the number of splice donor and splice acceptor sites. Because of the degeneracy of the genetic code, different nucleic acid molecules may encode the same protein. It is also well known that the genetic codes of different organisms are often biased towards using one of the several codons that encode the same amino acid over the others. Through codon optimization, changes are introduced in a nucleotide sequence that take advantage of the codon bias existing in a given cellular context so that the resulting codon optimized nucleotide sequence is more likely to be expressed in such given cellular context at a relatively high level compared to the non-codon optimized sequence. In a preferred embodiment of the invention, such optimized nucleotide sequence encoding a functional C-terminal truncated GDE polypeptide is codon-optimized to improve its expression in human cells compared to non-codon optimized nucleotide sequences coding for the same functional C-terminal truncated GDE polypeptide, for example by taking advantage of the human specific codon usage bias. The nucleic acid sequence encoding full-length human GDE isoform 1 is as shown in SEQ ID NO:4 or SEQ ID NO:58. Examples of corresponding codon optimized sequence is as shown in SEQ ID NO:5, SEQ ID NO:6, or SEQ ID NO:67.

In a particular embodiment, the nucleic acid molecule of the invention comprises or consists of the sequence shown in SEQ ID NO:56, encoding the functional C-terminal truncated GDE polypeptide having the amino acid sequence shown in SEQ ID NO:7.

The nucleic acid molecule encoding the functional C-terminal truncated GDE polypeptide as defined above may have at least 90 or at least 95 percent identity to the nucleotide sequence of SEQ ID NO:56.

In a particular embodiment, the nucleic acid molecule encoding the functional C-terminal truncated GDE polypeptide as defined above may have at least 90 or at least 95 percent identity to the nucleotide sequence of SEQ ID NO:56. In a particular embodiment, the nucleic acid molecule of the invention has at least 95 percent identity, for example at least 96, 97, 98, 99 or 100 percent identity to the nucleotide sequence of SEQ ID NO:56.

In a particular embodiment, the nucleic acid molecule of the invention encodes the functional C-terminal truncated GDE polypeptide having the amino acid sequence shown in SEQ ID NO:8.

In a particular embodiment, the nucleic acid molecule of the invention comprises or consists of the sequence shown in SEQ ID NO:57, encoding the functional C-terminal truncated GDE polypeptide having the amino acid sequence shown in SEQ ID NO:9.

The nucleic acid molecule encoding the functional C-terminal truncated GDE polypeptide as defined above may have at least 90 or at least 95 percent identity to the nucleotide sequence of SEQ ID NO:57. In a particular embodiment, the nucleic acid molecule encoding the functional C-terminal truncated GDE polypeptide as defined above may have at least 90 or at least 95 percent identity to the nucleotide sequence of SEQ ID NO:57. In a particular embodiment, the nucleic acid molecule of the invention has at least 95 percent identity, for example at least 96, 97, 98, 99 or 100 percent identity to the nucleotide sequence of SEQ ID NO:57.

The term “identical” and declinations thereof refers to the sequence identity between two nucleic acid molecules or between two polypeptide molecules. When a position in both of the two compared sequences is occupied by the same base or the same amino acid, then the molecules are identical at that position. The percent of identity between two sequences is a function of the number of matching positions shared by the two sequences divided by the number of positions compared X 100. For example, if 6 of 10 of the positions in two sequences are matched then the two sequences are 60% identical. Generally, a comparison is made when two sequences are aligned to give maximum identity. Various bioinformatics tools known to the one skilled in the art might be used to align nucleic acid sequences such as BLAST or FASTA.

As already mentioned, the above sequence may be codon-optimized.

In a particular embodiment, the nucleic acid molecule of the invention comprises or consists of:
the sequence shown in SEQ ID NO:34, encoding the functional truncated GDE polypeptide having the amino acid sequence shown in SEQ ID NO:25;
the sequence shown in SEQ ID NO:36, encoding the functional truncated GDE polypeptide having the amino acid sequence shown in SEQ ID NO:26;
the sequence shown in SEQ ID NO:37, encoding the functional truncated GDE polypeptide having the amino acid sequence shown in SEQ ID NO:27;
the sequence shown in SEQ ID NO:38, encoding the functional truncated GDE polypeptide having the amino acid sequence shown in SEQ ID NO:28;
the sequence shown in SEQ ID NO:40, encoding the functional truncated GDE polypeptide having the amino acid sequence shown in SEQ ID NO:29;
the sequence shown in SEQ ID NO:42, encoding the functional truncated GDE polypeptide having the amino acid sequence shown in SEQ ID NO:30;
the sequence shown in SEQ ID NO:43, encoding the functional truncated GDE polypeptide having the amino acid sequence shown in SEQ ID NO:31;
the sequence shown in SEQ ID NO:44, encoding the functional truncated GDE polypeptide having the amino acid sequence shown in SEQ ID NO:32;
the sequence shown in SEQ ID NO:45, encoding the functional truncated GDE polypeptide having the amino acid sequence shown in SEQ ID NO:33;
the sequence shown in SEQ ID NO:15 or SEQ ID NO:20, encoding the functional truncated GDE polypeptide having the amino acid sequence shown in SEQ ID NO:10;
the sequence shown in SEQ ID NO:16 or SEQ ID NO:21, encoding the functional truncated GDE polypeptide having the amino acid sequence shown in SEQ ID NO:11;
the sequence shown in SEQ ID NO:17 or SEQ ID NO:22, encoding the functional truncated GDE polypeptide having the amino acid sequence shown in SEQ ID NO:12;
the sequence shown in SEQ ID NO:18 or SEQ ID NO:23, encoding the functional truncated GDE polypeptide having the amino acid sequence shown in SEQ ID NO:13; or
the sequence shown in SEQ ID NO:19 or SEQ ID NO:24, encoding the functional truncated GDE polypeptide having the amino acid sequence shown in SEQ ID NO:14.

The nucleic acid molecule encoding the functional C-terminal truncated GDE polypeptide as defined above may have at least 90 or at least 95 percent identity to any of the nucleotide sequences of SEQ ID NO:34 to 45 and SEQ ID NO:15 to 24. In a particular embodiment, the nucleic acid molecule encoding the functional C-terminal truncated GDE polypeptide as defined above may have at least 90 or at least 95 percent identity to any of the nucleotide sequences of SEQ ID NO:34 to 45, SEQ ID NO:15 to 24. In a particular embodiment, the nucleic acid molecule of the invention has at least 95 percent identity, for example at least 96, 97, 98, 99 or 100 percent identity to any of the nucleotide sequences of SEQ ID NO:34 to 45 and SEQ ID NO:15 to 24.

3—Nucleic Acid Construct

The invention also relates to a nucleic acid construct comprising a nucleic acid molecule of the invention as described above. The nucleic acid construct may correspond to an expression cassette comprising the nucleic acid sequence of the invention, operably linked to one or more expression control sequences and/or other sequences improving the expression. As used herein, the term “operably linked” refers to a linkage of polynucleotide elements in a functional relationship. A nucleic acid is “operably linked” when it is placed into a functional relationship with another nucleic acid sequence. For instance, a promoter, or another transcription regulatory sequence, is operably linked to a coding sequence if it affects the transcription of the coding sequence. Such expression control sequences are known in the art, such as promoters, enhancers (such as cis-regulatory modules (CRMs)), introns, polyA signals, etc.

In a particular embodiment, the expression cassette may include a promoter. The promoter may be an ubiquitous or tissue-specific promoter, in particular a promoter able to promote expression in cells or tissues in which expression of GDE is desirable such as in cells or tissues in which GDE expression is desirable in GDE-deficient patients.

In a particular embodiment, the promoter is a muscle-specific promoter. Non-limiting examples of muscle-specific promoters include the muscle creatine kinase (MCK) promoter. Non-limiting examples of suitable muscle creatine kinase promoters are human muscle creatine kinase promoters and truncated murine muscle creatine kinase [(tMCK) promoters] (Wang B et al, Construction and analysis of compact muscle-selective promoters for AAV vectors. Gene Ther. 2008 November; 15(22):1489-99) (representative GenBank Accession No. AF188002). Human muscle creatine kinase has the Gene ID No. 1158 (representative GenBank Accession No. NC_000019.9, accessed on Dec. 26, 2012). Other examples of muscle-specific promoters include a synthetic promoter C5.12 (spC5.12, alternatively referred to herein as “C5.12”), such as the spC5.12 or the spC5.12 promoter (disclosed in Wang et al., Gene Therapy volume 15, pages 1489-1499 (2008)), the MHCK7 promoter (Salva et al. Mol Ther. 2007 February; 15(2):320-9), myosin light chain (MLC) promoters, for example MLC2 (Gene ID No. 4633; representative GenBank Accession No. NG_007554.1, accessed on Dec. 26, 2012); myosin heavy chain (MHC) promoters, for example alpha-MHC (Gene ID No. 4624; representative GenBank Accession No. NG 023444.1, accessed on Dec. 26, 2012); desmin promoters (Gene ID No. 1674; representative GenBank Accession No. NG_008043.1, accessed on Dec. 26, 2012); cardiac troponin C promoters (Gene ID No. 7134; representative GenBank Accession No. NG_008963.1, accessed on Dec. 26, 2012); troponin I promoters (Gene ID Nos. 7135, 7136, and 7137; representative GenBank Accession Nos. NG_016649.1, NG_011621.1, and NG_007866.2, accessed on Dec. 26, 2012); myoD gene family promoters (Weintraub et al., Science, 251, 761 (1991); Gene ID No. 4654; representative GenBank Accession No. NM_002478, accessed on Dec. 26, 2012); alpha actin promoters (Gene ID Nos. 58, 59, and 70; representative GenBank Accession Nos. NG_006672.1, NG_011541.1, and NG_007553.1, accessed on Dec. 26, 2012); beta actin promoters (Gene ID No. 60; representative GenBank Accession No. NG_007992.1, accessed on Dec. 26, 2012); gamma actin promoters (Gene ID No. 71 and 72; representative GenBank Accession No. NG_011433.1 and NM_001199893, accessed on Dec. 26, 2012); muscle-specific promoters residing within intron 1 of the ocular form of Pitx3 (Gene ID No. 5309) (Coulon et al; the muscle-selective promoter corresponds to residues 11219-11527 of representative GenBank Accession No. NG_008147, accessed on Dec. 26, 2012); and the promoters described in US Patent Publication US 2003/0157064, and CK6 promoters (Wang et al 2008 doi: 10.1038/gt.2008.104). In another particular embodiment, the muscle-specific promoter is the E-Syn promoter described in Wang et al., Gene Therapy volume 15, pages 1489-1499 (2008), comprising the combination of a MCK-derived enhancer and of the spC5.12 promoter. In a particular embodiment of the invention, the muscle-specific promoter is selected in the group consisting of a spC5.12 promoter, the MHCK7 promoter, the E-syn promoter, a muscle creatine kinase myosin light chain (MLC) promoter, a myosin heavy chain (MHC) promoter, a cardiac troponin C promoter, a troponin I promoter, a myoD gene family promoter, an alpha actin promoter, an beta actin promoter, an gamma actin promoter, a muscle-specific promoter residing within intron 1 of the ocular form of Pitx3, a CK6 promoter, a CK8 promoter and an Acta1 promoter. In a particular embodiment, the muscle-specific promoter is selected in the group consisting of the spC5.12, desmin and MCK promoters. In a further embodiment, the muscle-specific promoter is selected in the group consisting of the spC5.12 and MCK promoters. In a particular embodiment, the muscle-specific promoter is the spC5.12 promoter.

In a particular embodiment, the promoter is a liver-specific promoter. Non-limiting examples of liver-specific promoters include the alpha-1 antitrypsin promoter (hAAT), the transthyretin promoter, the albumin promoter, the thyroxine-binding globulin (TBG) promoter, the LSP promoter (comprising a thyroid hormone-binding globulin promoter sequence, two copies of an alpha1-microglobulin/bikunin enhancer sequence, and a leader sequence—Ill, C. R., et al. (1997). Optimization of the human factor VIII complementary DNA expression plasmid for gene therapy of hemophilia A. Blood Coag. Fibrinol. 8: S23-S30.), etc. Other useful liver-specific promoters are known in the art, for example those listed in the Liver Specific Gene Promoter Database compiled the Cold Spring Harbor Laboratory (http://rulai.cshl.edu/LSPD/). A preferred liver-specific promoter in the context of the invention is the hAAT promoter.

In another particular embodiment, the promoter is a neuron-specific promoter. Non-limiting examples of neuron-specific promoters include, but are not limited to the following: synapsin-1 (Syn) promoter, neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al. Neuron, 15:373-84 (1995)), among others which will be apparent to the skilled artisan. In a particular embodiment, the neuron-specific promoter is the Syn promoter. Other neuron-specific promoters include, without limitation: synapsin-2 promoter, tyrosine hydroxylase promoter, dopamine β-hydroxylase promoter, hypoxanthine phosphoribosyltransferase promoter, low affinity NGF receptor promoter, and choline acetyl transferase promoter (Bejanin et al., 1992; Carroll et al., 1995; Chin and Greengard, 1994; Foss-Petter et al., 1990; Harrington et al., 1987; Mercer et al., 1991; Patei et al., 1986). Representative promoters specific for the motor neurons include, without limitation, the promoter of the Calcitonin Gene-Related Peptide (CGRP), a known motor neuron-derived factor. Other promoters functional in motor neurons include the promoters of Choline Acetyl Transferase (ChAT), Neuron Specific Enolase (NSE), Synapsin and Hb9. Other neuron-specific promoters useful in the present invention include, without limitation: GFAP (for astrocytes), Calbindin 2 (for interneurons), Mnxl (motorneurons), Nestin (neurons), Parvalbumin, Somatostation and Plp1 (oligodendrocytes and Schwann cells).

In another particular embodiment, the promoter is a ubiquitous promoter. Representative ubiquitous promoters include the cytomegalovirus enhancer/chicken beta actin (CAG) promoter, the cytomegalovirus enhancer/promoter (CMV) (optionally with the CMV enhancer) [see, e.g., Boshart et al, Cell, 41:521-530 (1985)], the PGK promoter, the SV40 early promoter, the retroviral Rous sarcoma virus (RSV) LTR promoter (optionally with the RSV enhancer), the dihydrofolate reductase promoter, the β-actin promoter, the phosphoglycerol kinase (PGK) promoter, and the EF1 alpha promoter.

In addition, the promoter may also be an endogenous promoter such as the albumin promoter or the GDE promoter.

In a particular embodiment, the promoter is associated to an enhancer sequence, such as a cis-regulatory module (CRMs) or an artificial enhancer sequence. CRMs useful in the practice of the present invention include those described in Rincon et al., Mol Ther. 2015 January; 23(1):43-52, Chuah et al., Mol Ther. 2014 September; 22(9):1605-13 or Nair et al., Blood. 2014 May 15; 123(20):3195-9. Other regulatory elements that are, in particular, able to enhance muscle-specific expression of genes, in particular expression in cardiac muscle and/or skeletal muscle, are those disclosed in WO2015110449. Particular examples of nucleic acid regulatory elements that comprise an artificial sequence include the regulatory elements that are obtained by rearranging the transcription factor binding sites (TFBS) that are present in the sequences disclosed in WO2015110449. Said rearrangement may encompass changing the order of the TFBSs and/or changing the position of one or more TFBSs relative to the other TFBSs and/or changing the copy number of one or more of the TFBSs. For example, a nucleic acid regulatory element for enhancing muscle-specific gene expression, in particular cardiac and skeletal muscle-specific gene expression, may comprise binding sites for E2A, HNH 1, NF1, C/EBP, LRF, MyoD, and SREBP; or for E2A, NF1, p53, C/EBP, LRF, and SREBP; or for E2A, HNH 1, HNF3a, HNF3b, NF1, C/EBP, LRF, MyoD, and SREBP; or E2A, HNF3a, NF1, C/EBP, LRF, MyoD, and SREBP; or for E2A, HNF3a, NF1, CEBP, LRF, MyoD, and SREBP; or for HNF4, NF1, RSRFC4, C/EBP, LRF, and MyoD, or NF1, PPAR, p53, C/EBP, LRF, and MyoD. For example, a nucleic acid regulatory element for enhancing muscle-specific gene expression, in particular skeletal muscle-specific gene expression, may also comprise binding sites for E2A, NF1, SRFC, p53, C/EBP, LRF, and MyoD; or for E2A, NF1, C/EBP, LRF, MyoD, and SREBP; or for E2A, HNF3a, C/EBP, LRF, MyoD, SEREBP, and Tall_b; or for E2A, SRF, p53, C/EBP, LRF, MyoD, and SREBP; or for HNF4, NF1, RSRFC4, C/EBP, LRF, and SREBP; or for E2A, HNF3a, HNF3b, NF1, SRF, C/EBP, LRF, MyoD, and SREBP; or for E2A, CEBP, and MyoD. In further examples, these nucleic acid regulatory elements comprise at least two, such as 2,3,4, or more copies of one or more of the TFBSs recited before. Other regulatory elements that are, in particular, able to enhance liver-specific expression of genes, are those disclosed in WO2009130208. In another particular embodiment, the nucleic acid construct comprises an intron, in particular an intron placed between the promoter and the GDE coding sequence. An intron may be introduced to increase mRNA stability and the production of the protein. In a further embodiment, the intron is a human beta globin b2 (or HBB2) intron, a coagulation factor IX (FIX) intron, a SV40 intron, a hCMV intron A (hCMVI), a TPL intron (TPLI), a CHEF1 gene intron1 (CHEFI), a MVM intron (Wu et al, 2008), a FIX truncated intron 1 (Wu et al., 2008, Mol Ther, 16(2):280-289; Kurachi et al., 1995, J Biol Chem., 270(10):5276-5281), a β-globin/immunoglobin heavy chain hybrid intron (5′-donor site from a human β-globin intron and 3′-acceptor site from an immunoglobulin heavy chain variable region intron, Wu et al., 2008, Mol Ther, 16(2):280-289; Kurachi et al., 1995, J Biol Chem., 270(10):5276-5281), a hybrid intron consisting of an adenovirus splice donor and an immunoglobulin G splice (Wong et al., 1985, Chromosoma, 92(2):124-135; Yew et al., 1997, Hum Gene Ther, 8(5):575-584; Choi T. et al., 1991, Mol Cell Biol, 11(6):3070-3074; Huang et al., 1990, Mol Cell Biol., 10(4):1805-1810), a hybrid 19S/16S SV40 intron (5′-donor site from 19S intron and 3′-acceptor site from 16S intron, Yew et al., 1997, Hum Gene Ther, 8(5):575-584) or a chicken beta-globin intron. In another further embodiment, the intron is a modified intron (in particular a modified HBB2 or FIX intron) designed to decrease the number of, or even totally remove, alternative open reading frames (ARFs) found in said intron. Preferably, ARFs are removed whose length spans over 50 bp and have a stop codon in frame with a start codon. ARFs may be removed by modifying the sequence of the intron. For example, modification may be carried out by way of nucleotide substitution, insertion or deletion, preferably by nucleotide substitution. As an illustration, one or more nucleotides, in particular one nucleotide, in an ATG or GTG start codon present in the sequence of the intron of interest may be replaced resulting in a non-start codon. For example, an ATG or a GTG may be replaced by a CTG, which is not a start codon, within the sequence of the intron of interest.

The classical HBB2 intron is shown in SEQ ID NO:46. For example, this HBB2 intron may be modified by eliminating start codons (ATG and GTG codons) within said intron. In a particular embodiment, the modified HBB2 intron has the sequence shown in SEQ ID NO:47. The classical FIX intron is derived from the first intron of human FIX and is shown in SEQ ID NO:48. FIX intron may be modified by eliminating start codons (ATG and GTG codons) within said intron. In a particular embodiment, the modified FIX intron has the sequence shown in SEQ ID NO:49. The classical chicken-beta globin intron used in nucleic acid constructs is shown in SEQ ID NO:50. Chicken-beta globin intron may be modified by eliminating start codons (ATG and GTG codons) within said intron. In a particular embodiment, the modified chicken-beta globin intron has the sequence shown in SEQ ID NO:51.

The inventors have previously shown in WO2015/162302 that such a modified intron, in particular a modified HBB2 or FIX intron, has advantageous properties and can significantly improve the expression of a transgene.

In a particular embodiment, the nucleic acid construct of the invention is an expression cassette comprising, in the 5′ to 3′ orientation, a promoter optionally preceded by an enhancer, the coding sequence of the invention (i.e. the nucleic acid molecule encoding the functional C-terminal truncated GDE polypeptide), and a polyadenylation signal such as the bovine growth hormone polyadenylation signal (bGH polyA), the SV40 polyadenylation signal, or another naturally occurring or artificial polyadenylation signal. Preferably, the polyadenylation signal is the bGH polyA. In a particular embodiment, the polyadenylation signal is the bGH poly A as shown in SEQ ID NO:53. In a particular embodiment, a very short polyA signal is used. For example, a very short polyA signal comprising less than 20 nucleotides is used. In a particular embodiment, the polyadenylation signal is the human soluble neuropilin-1 (sNRP) polyadenylation signal (sNRP polyA; SEQ ID NO:52). In another particular embodiment, the polyadenylation signal is the pA58 polyadenylation signal as shown in SEQ ID NO:54.

In a particular embodiment, the nucleic acid construct of the invention is an expression cassette comprising, in the 5′ to 3′ orientation, a promoter optionally preceded by an enhancer, an intron, the coding sequence of the invention, and a polyadenylation signal. In another embodiment, the nucleic acid construct of the invention is an expression cassette comprising, in the 5′ to 3′ orientation, a promoter, the coding sequence of the invention, and a polyadenylation signal. In another embodiment, the nucleic acid construct of the invention is an expression cassette comprising, in the 5′ to 3′ orientation, an enhancer, a promoter, the coding sequence of the invention, and a polyadenylation signal. In another embodiment, the nucleic acid construct of the invention is an expression cassette comprising, in the 5′ to 3′ orientation, a SpC5-12 promoter or a CMV promoter such as a mini-CMV promoter, the coding sequence of the invention, and a polyadenylation signal (such as a bGH polyA or a sNRP polyA, in particular a bGH polyA). In another embodiment, the nucleic acid construct of the invention is an expression cassette comprising, in the 5′ to 3′ orientation, an enhancer, a SpC5-12 promoter or a CMV promoter such as a mini-CMV promoter, the coding sequence of the invention, and a polyadenylation signal (such as a bGH polyA or a sNRP polyA, in particular a bGH polyA). In a further particular embodiment, the nucleic acid construct of the invention is an expression cassette comprising, in the 5′ to 3′ orientation, an enhancer, a promoter, an intron, the coding sequence of the invention, and a polyadenylation signal. In a further particular embodiment of the invention the expression cassette comprising, in the 5′ to 3′ orientation a promoter, an optional intron, the coding sequence of the invention and a polyA signal. In a further particular embodiment, the expression cassette comprises, in the 5′ to 3′ orientation: a SpC5-12 promoter; a SV40 intron; a sequence coding the amino acid sequence of SEQ ID NO:7, SEQ ID NO:8, SEQ ID NO:9, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:10, SEQ ID NO:11, SEQ ID NO:12, SEQ ID NO:13, SEQ ID NO:14, SEQ ID NO:64, SEQ ID NO:65 or SEQ ID NO:66, in particular SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:64, SEQ ID NO:65 or SEQ ID NO:66, in particular SEQ ID NO:7, SEQ ID NO:8 or SEQ ID NO:9, more particularly SEQ ID NO:7; and a bGH polyA. In a further particular embodiment, the nucleic acid construct of the invention is an expression cassette comprising, in the 5′ to 3′ orientation, a promoter, the coding sequence of the invention, and a polyadenylation signal. In a further particular embodiment of the invention the expression cassette comprising, in the 5′ to 3′ orientation an enhancer, a promoter, the coding sequence of the invention and a polyA signal. In a further particular embodiment, the expression cassette comprises, in the 5′ to 3′ orientation: a SpC5-12 promoter; a sequence coding the amino acid sequence of SEQ ID NO:7, SEQ ID NO:8, SEQ ID NO:9, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:10, SEQ ID NO:11, SEQ ID NO:12, SEQ ID NO:13 or SEQ ID NO:14, SEQ ID NO:64, SEQ ID NO:65 or SEQ ID NO:66, in particular SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:64, SEQ ID NO:65 or SEQ ID NO:66, in particular SEQ ID NO:7, SEQ ID NO:8 or SEQ ID NO:9, more particularly SEQ ID NO:7; and a bGH polyA or sNRP polyA, in particular a bGH polyA. In another embodiment, the expression cassette comprises, in the 5′ to 3′ orientation: a CMV promoter; a SV40 intron; a sequence coding the amino acid sequence of SEQ ID NO:7, SEQ ID NO:8, SEQ ID NO:9, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:10, SEQ ID NO:11, SEQ ID NO:12, SEQ ID NO:13, SEQ ID NO:14, SEQ ID NO:64, SEQ ID NO:65 or SEQ ID NO:66, in particular SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:64, SEQ ID NO:65 or SEQ ID NO:66, in particular SEQ ID NO:7, SEQ ID NO:8 or SEQ ID NO:9, more particularly SEQ ID NO:7; and a bGH polyA. In another embodiment, the expression cassette comprises, in the 5′ to 3′ orientation: a CMV promoter; a sequence coding the amino acid sequence of SEQ ID NO:7, SEQ ID NO:8, SEQ ID NO:9, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:10, SEQ ID NO:11, SEQ ID NO:12, SEQ ID NO:13, SEQ ID NO:14, SEQ ID NO:64, SEQ ID NO:65 or SEQ ID NO:66, in particular SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:64, SEQ ID NO:65 or SEQ ID NO:66, in particular SEQ ID NO:7, SEQ ID NO:8 or SEQ ID NO:9, more particularly SEQ ID NO:7; and a bGH polyA or sNRP polyA, in particular a bGH polyA.

In a particular embodiment, the expression cassette comprises in the 5′ to 3′ orientation: a CMV promoter such as a mini-CMV promoter; a sequence encoding the functional C-terminal truncated GDE polypeptide as defined above such as a sequence coding the amino acid sequence of SEQ ID NO:7, SEQ ID NO:8, SEQ ID NO:9, in particular SEQ ID NO:7; and a bGH polyA or sNRP polyA, in particular a bGH polyA.

In a particular embodiment, the expression cassette comprises or consists of the sequence as shown in SEQ ID NO:55.

In designing the nucleic acid construct of the invention, one skilled in the art will take care of respecting the size limit of the vector used for delivering said construct to a cell or organ. In particular, one skilled in the art knows that a major limitation of AAV vector is its cargo capacity which may vary from one AAV serotype to another but is thought to be limited to around the size of parental viral genome. For example, 5 kb, is the maximum size usually thought to be packaged into an AAV8 capsid (Wu Z. et al., Mol Ther., 2010, 18(1): 80-86; Lai Y. et al., Mol Ther., 2010, 18(1): 75-79; Wang Y. et al., Hum Gene Ther Methods, 2012, 23(4): 225-33). In addition, during recombinant AAV production, genomes larger than 5 kb are encapsidated with low efficacy and the resulting AAV may contain fragmented genomes reducing the efficacy of gene transfer. Accordingly, those skilled in the art will take care in practicing the present invention to select the components of the nucleic acid construct of the invention so that the resulting nucleic acid sequence, including sequences coding AAV 5′- and 3′-ITRs to preferably not exceed 110% of the cargo capacity of the AAV vector implemented, in particular to preferably not exceed 5 kb. AAV vectors having larger cargo capacity can also be used in the context on the present invention. For example AAV particles lacking Vp2 subunit are shown to successfully package larger genomes (i.e. 6 kb) while preserving integrity of encapsidated genomes (Grieger et al., 2005, J Virol., 79(15):9933-9944).

4—Vector

The present invention also relates to a vector comprising a nucleic acid molecule or construct as disclosed herein. In a particular embodiment, the vector comprises a nucleic acid molecule or construct encoding a functional C-terminal truncated GDE polypeptide as defined above.

In particular, the vector of the invention is a vector suitable for protein expression, preferably for use in gene therapy. In one embodiment, the vector is a plasmid vector. In another embodiment, the vector is a nanoparticle containing a nucleic acid molecule of the invention, in particular a messenger RNA encoding the functional C-terminal truncated GDE polypeptide of the invention. In another embodiment, the vector is a system based on transposons, allowing integration of the nucleic acid molecule or construct of the invention in the genome of the target cell, such as the hyperactive Sleeping Beauty (SB100X) transposon system (Mates et al. 2009). In another embodiment, the vector is a viral vector suitable for gene therapy, targeting any cell of interest such as liver tissue or cells, muscle cell, CNS cells (such as brain cells), or hematopoietic stem cells such as cells of the erythroid lineage (such as erythrocytes). In this case, the nucleic acid construct of the invention also contains sequences suitable for producing an efficient viral vector, as is well known in the art.

Viral vectors are preferred for delivering the nucleic acid molecule or construct of the invention, such as a retroviral vector, for example a lentiviral vector, or a non-pathogenic parvovirus, more preferably an AAV vector. The human parvovirus Adeno-Associated Virus (AAV) is a dependovirus that is naturally defective for replication which is able to integrate into the genome of the infected cell to establish a latent infection. The last property appears to be unique among mammalian viruses because the integration occurs at a specific site in the human genome, called AAVS1, located on chromosome 19 (19q13.3-qter).

Therefore, AAV vectors have arisen considerable interest as potential vectors for human gene therapy. Among the favorable properties of the virus are its lack of association with any human disease, its ability to infect both dividing and non-dividing cells, and the wide range of cell lines derived from different tissues that can be infected.

Among the serotypes of AAVs isolated from human or non-human primates (NHP) and well characterized, human serotype 2 is the first AAV that was developed as a gene transfer vector. Other currently used AAV serotypes include AAV-1, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods.), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9, -2G9, -10 such as cy10 and -rh10, -rh74, -dj, Anc80, LK03, AAV2i8, porcine AAV serotypes such as AAVpo4 and AAVpo6, and tyrosine, lysine and serine capsid mutants of the AAV serotypes, etc. In addition, other non-natural engineered variants and chimeric AAV can also be useful. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells.

AAV-based recombinant vectors lacking the Rep protein integrate with low efficacy into the host's genome and are mainly present as stable circular episomes that can persist for years in the target cells. Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

In the context of the present invention, the AAV vector comprises an AAV capsid able to transduce the target cells of interest, i.e. cells of the tolerogenic tissue (for example hepatocytes) and cells of the tissue(s) of therapeutic interest such as muscle cells, CNS cells or cardiac cells. In a particular embodiment, the AAV vector comprises an AAV capsid able to transduce muscle cells or cardiac cells.

According to a particular embodiment, the AAV vector is of the AAV-1, -2, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9, -9P1, -2G9, -10 such as -cy10 and -rh10, -rh39, -rh43, -rh74, -dj, Anc80, LK03, AAV.PHP, AAV2i8, porcine AAV such as AAVpo4 and AAVpo6, and tyrosine, lysine and serine capsid mutants of AAV serotypes. In a particular embodiment, the AAV vector is of the AAV6, AAV8, AAV9, AAV9P1, AAVrh74 or AAV2i8 serotype (i.e. the AAV vector has a capsid of the AAV6, AAV8, AAV9, AAV9P1, AAVrh74 or AAV2i8 serotype). In a further particular embodiment, the AAV vector is a pseudotyped vector, i.e. its genome and capsid are derived from AAVs of different serotypes. For example, the pseudotyped AAV vector may be a vector whose genome is derived from one of the above mentioned AAV serotypes, and whose capsid is derived from another serotype. For example, the genome of the pseudotyped vector may have a capsid derived from the AAV6, AAV8, AAV9, AAV9P1, AAVrh74 or AAV2i8 serotype, and its genome may be derived from and different serotype. In a particular embodiment, the AAV vector has a capsid of the AAV6, AAV8, AAV9 or AAVrh74 serotype, in particular of the AAV6, AAV8, AAV9, or AAV9P1 serotype, more particularly of the AAV6, AAV9 or AAV9P1 serotype.

In a specific embodiment, wherein the vector is for use in delivering the therapeutic transgene to muscle cells, the AAV vector may be selected, among others, in the group consisting of AAV8, AAV9 and AAVrh74.

In another specific embodiment, wherein the vector is for use in delivering the transgene to liver cells, the AAV vector may be selected, among others, in the group consisting of AAV1, AAVS, AAV8, AAV9, AAVrh10, AAVrh39, AAVrh43, AAVrh74, AAV-LK03, AAV2G9, AAV.PHP, AAV-Anc80 and AAV3B.

In a further specific embodiment, wherein the vector is for use in delivering the transgene to the CNS, the AAV vector may be selected, among others, in the group consisting of AAV9, AAV9P1, AAV10 and AAV2G9.

In another embodiment, the capsid is a modified capsid. In the context of the present invention, a “modified capsid” may be a chimeric capsid or capsid comprising one or more variant VP capsid proteins derived from one or more wild-type AAV VP capsid proteins.

In a particular embodiment, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. Examples of such chimeric AAV vectors useful to transduce liver cells are described in Shen et al., Molecular Therapy, 2007 and in Tenney et al., Virology, 2014. For example, a chimeric AAV vector can derive from the combination of an AAV8 capsid sequence with a sequence of an AAV serotype different from the AAV8 serotype, such as any of those specifically mentioned above. In another embodiment, the capsid of the AAV vector comprises one or more variant VP capsid proteins such as those described in WO2015013313, in particular the RHM4-1, RHM15-1, RHM15-2, RHM15-3/RHM15-5, RHM15-4 and RHM15-6 capsid variants, which present a high liver tropism.

In another embodiment, the modified capsid can be derived from capsid modifications inserted by error prone PCR and/or peptide insertion (e.g. as described in Bartel et al., 2011). In a particular embodiment, the capsid includes the P1 modification, as described in PCT/EP2019/058560. In addition, capsid variants may include single amino acid changes such as tyrosine mutants (e.g. as described in Zhong et al., 2008). In a particular embodiment, the vector is an AAVrh74 comprising the P1 insertion.

In a particular embodiment, the AAV vector is an AAV vector as described in EP2019/058560 or an AAV vector as described in EP2019/076958.

In addition, the genome of the AAV vector may either be a single stranded or self-complementary double-stranded genome (McCarty et al., Gene Therapy, 2003). Self-complementary double-stranded AAV vectors are generated by deleting the terminal resolution site from one of the AAV terminal repeats. These modified vectors, whose replicating genome is half the length of the wild type AAV genome have the tendency to package DNA dimers. In a preferred embodiment, the AAV vector implemented in the practice of the present invention has a single stranded genome, and further preferably comprises an AAV8, AAV9, AAVrh74 or AAV2i8 capsid, in particular an AAV8, AAV9 or AAVrh74 capsid, such as an AAV8 or AAV9 capsid, more particularly an AAV9 capsid.

The AAV vector used for packaging the GDE sequence of the invention can also be modified in order to increase its cargo capacity. For example, AAV vectors lacking Vp2 subunit are shown to successfully package larger genomes (i.e. 6 kb) while preserving integrity of encapsidated genomes (Grieger et al., 2005).

As is known in the art, additional suitable sequences may be introduced in the nucleic acid construct of the invention for obtaining a functional viral vector. Suitable sequences include AAV ITRs.

In a particular embodiment, the AAV vector comprises a muscle-specific promoter as described above, in particular a muscle-specific promoter that presents some leakage of expression into liver cells.

In another particular embodiment of the invention, the AAV vector comprises a liver-specific promoter as described above. The protolerogenic and metabolic properties of the liver are advantageously implemented thanks to this embodiment to develop highly efficient and optimized vectors to express GDE in hepatocytes and to induce immune tolerance to the protein.

In a particular embodiment, a dual recombinant AAV vector system comprising two AAV vectors as described in WO 2018/162748 is used for delivering the nucleic acid molecule or construct encoding the functional C-terminal truncated GDE polypeptide as defined above. In particular, the dual AAV vector system comprises:
a first AAV vector comprising, between 5′ and 3′ AAV ITRs, a first nucleic acid sequence that encodes a N-terminal part of the C-terminal truncated GDE polypeptide as defined above, and
a second AAV vector comprising, between 5′ and 3′ AAV ITRs, a second nucleic acid sequence that encodes a C-terminal part of the C-terminal truncated GDE polypeptide as defined above, and wherein the first and second nucleic acid sequences encoding said GDE comprise a polynucleotide region that permits the production of a full-length C-terminal truncated GDE polypeptide as defined above.

In another particular embodiment, the AAV vector is a single AAV vector comprising a nucleic acid molecule or construct encoding a functional C-terminal truncated GDE polypeptide as defined above.

5—Cell

The invention also relates to a cell, in particular an isolated cell, for example a liver cell, a cardiac cell, a CNS cell or a muscle cell, that is transformed or transduced with the nucleic acid molecule, the construct or the vector of the invention. In a particular embodiment, the cell is an isolated human cell. In a further particular embodiment, the cell is not a human embryonic stem cell. The cell of the invention expresses a functional C-terminal truncated GDE polypeptide as described above. Cells of the invention may be delivered to the subject in need thereof, such as GDE-deficient patient, by any appropriate administration route such as via injection in the liver, in the CNS, in the heart, in the muscle(s) or in the bloodstream of said subject. In a particular embodiment, the invention involves transducing liver or muscle cells, in particular liver or muscle cells of the subject to be treated, and administering said transduced liver and/or muscle cells into which the nucleic acid has been introduced to the subject. In a particular embodiment, the liver cells are liver cells from the patient to be treated, or are liver stem cells that are further transformed, and differentiated in vitro into liver cells, for subsequent administration to the patient. In another embodiment, the cell is a muscle cell from the patient to be treated, or is a muscle stem cell that is further transformed, and optionally differentiated in vitro into muscle cells, for subsequent administration to the patient.

6—Pharmaceutical Composition

The present invention also provides pharmaceutical compositions comprising the nucleic acid molecule, the nucleic acid construct, the vector, the functional C-terminal truncated GDE polypeptide, or the cell of the invention. Such compositions may comprise a therapeutically effective amount of the therapeutic (the nucleic acid molecule, the nucleic acid construct, the vector, the functional C-terminal truncated GDE polypeptide or the cell of the invention), and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject. In a particular embodiment, the nucleic acid, vector or cell of the invention is formulated in a composition comprising phosphate-buffered saline and supplemented with 0.25% human serum albumin. In another particular embodiment, the nucleic acid, vector or cell of the invention is formulated in a composition comprising ringer lactate and a non-ionic surfactant, such as pluronic F68 at a final concentration of 0.01-0.0001%, such as at a concentration of 0.001%, by weight of the total composition. The formulation may further comprise serum albumin, in particular human serum albumin, such as human serum albumin at 0.25%. Other appropriate formulations for either storage or administration are known in the art, in particular from WO 2005/118792 or Allay et al., 2011.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection.

In an embodiment, the nucleic acid molecule, the nucleic acid construct, the vector, the functional C-terminal truncated GDE polypeptide or the cell of the invention can be delivered in a vesicle, in particular a liposome. In yet another embodiment, the nucleic acid molecule, the nucleic acid construct, the vector, the functional C-terminal truncated GDE polypeptide or the cell of the invention can be delivered in a controlled release system.

In a particular embodiment, the nucleic acid molecule is delivered as a mRNA, corresponding to the transcript encoding the functional C-terminal truncated GDE polypeptide of the invention. In particular, the mRNA of the invention may be delivered using liposomes such as lipid nanoparticle (LNP).

Methods of administration of the nucleic acid molecule, the nucleic acid construct, the vector, the functional C-terminal truncated polypeptide or the cell of the invention include but are not limited to intradermal, intramuscular, intraperitoneal, intravenous, subcutaneous, intranasal, epidural, and oral routes. In a particular embodiment, the administration is via the intravenous or intramuscular route. The nucleic acid molecule, the nucleic acid construct, the vector, the functional C-terminal truncated GDE polypeptide or the cell of the invention, whether vectorized or not, may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment, e.g. the liver or the muscle. This may be achieved, for example, by means of an implant, said implant being of a porous, nonporous, or gelatinous material, including membranes, such as sialastic membranes, or fibers.

In a particular embodiment, the functional C-terminal truncated GDE polypeptide of the invention is used in enzyme replacement therapy (ERT), in particular for treating GSDIII. The term “enzyme replacement therapy” or “ERT” generally refers to the introduction of a purified enzyme into an individual having a deficiency in such enzyme. The administered polypeptide of the invention can be obtained from natural sources, by recombinant expression, produced in vitro, or purified from isolated tissue or fluid. In particular, when used in ERT, the polypeptide of the invention may be administered parenterally, such as via intraperitoneal, intramuscular, intravascular (i.e. intravenous or intraarterial) administration. In particular the polypeptide is administered by intravenous injection. Said administration may be repeated frequently, such as every day, every week, every two weeks or every month, in particular every week or every two weeks.

The amount of the therapeutic (i.e. the nucleic acid molecule, the nucleic acid construct, the vector, the functional C-terminal truncated GDE polypeptide or the cell of the invention) of the invention which will be effective in the treatment of GSDIII can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage of the nucleic acid molecule, the nucleic acid construct, the vector, the functional C-terminal truncated GDE polypeptide or the cell of the invention administered to the subject in need thereof will vary based on several factors including, without limitation, the route of administration, the specific disease treated, the subject's age or the level of expression necessary to achieve the therapeutic effect. One skilled in the art can readily determine, based on its knowledge in this field, the dosage range required based on these factors and others. In case of a treatment comprising administering a viral vector, such as an AAV vector, to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1 Δ10¹² vg/kg at least 1×10¹³ vg/kg, or at least 1×10¹⁴ vg/kg.

7—Method of Treatment

The invention also relates to a method for treating GSDIII, which comprises a step of delivering a therapeutic effective amount of the nucleic acid molecule, the nucleic acid construct, the vector, the functional C-terminal truncated GDE polypeptide, the pharmaceutical composition or the cell of the invention to a subject in need thereof.

Cirrhosis and hepatocellular carcinoma can also develop in patients with GSDIII. Thus, the invention also relates to a method for treating cirrhosis and hepatocellular carcinoma in a GSDIII patient which comprises a step of delivering a therapeutic effective amount of the nucleic acid molecule, the nucleic acid construct, the vector, the functional C-terminal truncated GDE polypeptide, the pharmaceutical composition or the cell of the invention to a subject in need thereof.

The invention also relates to a method for treating GSDIII, said method inducing no immune response to the transgene (i.e. to the functional C-terminal truncated GDE polypeptide encoded by the nucleic acid molecule), or inducing a reduced immune response to the transgene, comprising a step of delivering a therapeutic effective amount of the nucleic acid, the vector, the functional C-terminal truncated GDE polypeptide, the pharmaceutical composition or the cell of invention to a subject in need thereof. The invention also relates to a method for treating GSDIII, said method comprising repeated administration of a therapeutic effective amount of the nucleic acid, the vector, the functional C-terminal truncated GDE polypeptide, the pharmaceutical composition or the cell of the invention to a subject in need thereof. In this aspect, the nucleic acid molecule, the nucleic acid construct or the vector of the invention comprises a promoter which is functional in liver cells, thereby allowing immune tolerance to the expressed functional C-terminal truncated GDE polypeptide produced therefrom. As well, in this aspect, the pharmaceutical composition used in this aspect comprises a nucleic acid molecule, a nucleic acid construct or a vector comprising a promoter which is functional in liver cells. In case of delivery of cells, in particular of liver, cardiac, CNS or muscle cells, said cells may be cells previously collected from the subject in need of the treatment and that were engineered by introducing therein the nucleic acid molecule, the nucleic acid construct or the vector of the invention to thereby make them able to produce the functional C-terminal truncated GDE polypeptide. According to an embodiment, in the aspect comprising a repeated administration, said administration may be repeated at least once or more, and may even be considered to be done according to a periodic schedule, such as once per week, per month or per year. The periodic schedule may also comprise an administration once every 2, 3, 4, 5, 6, 7, 8, 9 or 10 year, or more than 10 years. In another particular embodiment, administration of each administration of a viral vector of the invention is done using a different virus for each successive administration, thereby avoiding a reduction of efficacy because of a possible immune response against a previously administered viral vector. For example, a first administration of an AAV vector comprising an AAV8 capsid may be done, followed by the administration of a vector comprising an AAV9 capsid.

According to the present invention, a treatment may include curative, alleviation or prophylactic effects. Accordingly, therapeutic and prophylactic treatment includes amelioration of the symptoms of GSDIII or preventing or otherwise reducing the risk of developing a particular glycogen storage disease. The term “prophylactic” may be considered as reducing the severity or the onset of a particular condition. “Prophylactic” also includes preventing reoccurrence of a particular condition in a patient previously diagnosed with the condition. “Therapeutic” may also reduce the severity of an existing condition. The term “treatment” is used herein to refer to any regimen that can benefit an animal, in particular a mammal, more particularly a human subject.

The invention also relates to an ex vivo gene therapy method for the treatment of GSDIII, comprising introducing the nucleic acid molecule, the nucleic acid construct or the vector of the invention into an isolated cell of a patient in need thereof, for example an isolated hematopoietic stem cell, and introducing said cell into said patient in need thereof.

The invention also relates to the nucleic acid molecule, the nucleic acid construct, the vector, the functional C-terminal truncated GDE polypeptide, the cell or the pharmaceutical composition of the invention for use as a medicament.

The invention also relates to the nucleic acid molecule, the nucleic acid construct, the vector, the functional C-terminal truncated GDE polypeptide, the cell or the pharmaceutical composition of the invention, for use in a method for treating a disease caused by a mutation in the AGL gene encoding GDE, in particular in a method for treating GSDIII (Cori disease), such as GSDIIIa and GSDIIIb, in particular GSDIIIa.

The invention further relates to the use of the nucleic acid molecule, the nucleic acid construct, the vector, the functional C-terminal truncated GDE polypeptide, the cell or the pharmaceutical composition of the invention, in the manufacture of a medicament useful for treating GSDIII (Cori disease).

The invention also relates to the nucleic acid molecule, the nucleic acid construct, the vector, the functional C-terminal truncated GDE polypeptide, the cell or the pharmaceutical composition of the invention, for use in a method for delivering the GDE protein in the affected tissues, in particular in muscle and liver tissues, in particular in muscles.

### EXAMPLES

The invention is further described in detail by reference to the following experimental examples and the attached figures. These examples are provided for purposes of illustration only, and are not intended to be limiting.

### 1—Example 1

Material and Methods

To evaluate the activity of the C-terminal truncated GDE (ΔCter-1, SEQ ID NO:7), AAV vectors expressing the ΔCter-1-GDE (AAV-ΔCter-1-GDE) or the full-size protein (AAV-FS-GDE) were produced. The transgene was cloned in a transgene expression cassette optimized for muscle expression and composed of a mini CMV promoter and a bGH poly adenylation signal. The two vectors were injected in 4-6 month-old GSDIII mice at the dose of 2 Δ10¹² vg/mouse. PBS-injected wild-type (WT, PBS) or knockout mice (KO, PBS) were used as controls. The measurement of muscle function by wire-hang was performed one week before (D-7) and 5 weeks after (D35) vectors injection, as described below. Mice injected with AAV vectors expressing C-terminal truncated GDE or full size GDE were sacrificed 5 weeks after injections for measuring Glycogen content in heart and quadriceps.

Measurement of Glycogen Content

Glycogen content was measured indirectly in tissue homogenates as the glucose released after total digestion with Aspergillus Niger amyloglucosidase (Sigma Aldrich, Saint Louis, MO). Samples were incubated for 5 min at 95° C. and then cooled at 4° C.; 25 μl of amyloglucosidase diluted 1:50 in 0.1M potassium acetate pH5.5 were then added to each sample. A control reaction without amyloglucosidase was prepared for each sample. Both sample and control reactions were incubated at 37° C. for 90 minutes. The reaction was stopped by incubating samples for 5 min at 95° C. The glucose released was determined with a commercial glucose assay kit (Sigma Aldrich, Saint Louis, MO) and the resulting absorbance was acquired on an EnSpire alpha plate reader (Perkin-Elmer, Waltham, MA) at a wavelength of 540 nm.

Muscle Function Tests

To measure the mean hanging time, a three-minute lasting hanging test on a 4-mm wire was performed. At the beginning of the test, a “falling” score of 10 is attributed to each animal. A mouse is handled by 35 the tail and brought near the wire. The operator suspends the animal by the fore limbs only. As soon as the animal is properly suspended, a 180-sec timer is started. If the animal falls, the timer is stopped, the falling score is diminished by 1 and the elapsed time is noted. The animal is then suspended by the fore limbs and the timer started again. The test is stopped either when the timer or the falling score reach 0. Results are expressed as number of falls per minute.

### Results

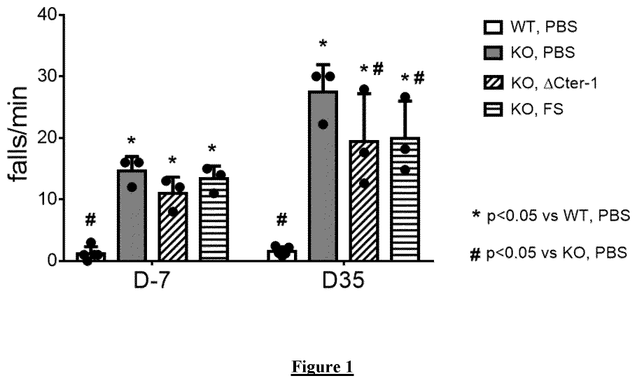

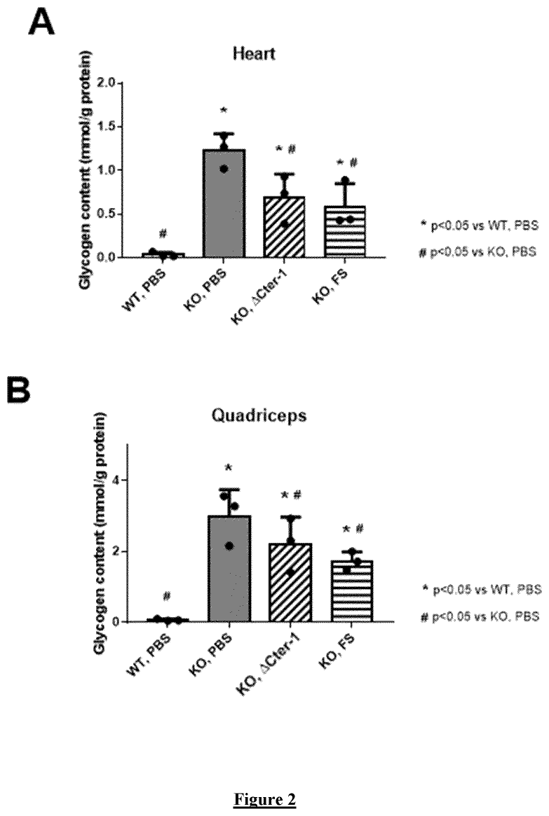

The measurement of muscle function by wire-hang was performed one week before (D-7) and 5 weeks after (D35) vectors injection. AAV-treated mice showed a partial reduction in the number of falls per minute (FIG. 1). Glycogen accumulation was then evaluated in heart and quadriceps of GDE-KO animals treated with the AAV vectors. Interestingly, significant reduction of glycogen content was measured in heart and quadriceps of mice treated with the two vectors (FIG. 2).

### 2—Example 2

Material and Methods

Activity of the C-terminal truncated GDE (ΔCter-1) was also evaluated over a longer period, by assessing wire-hang performance 3 months post-injection. For this experiment, GDE knockout (KO) mice were injected with PBS (KO_PBS); with 3.3×10¹² vg/mouse or 9.9×10¹² vg/mouse (High dose) of an AAV vector encoding C-terminal truncated GDE (ΔCter-1); or with 3.3×10¹² vg/mouse of an AAV vector encoding full-size GDE (GDE FS). PBS-injected wild-type mice were used as controls (WT_PBS). Wire-hang performance was measured one week before (D-7) and 3 months after vectors injection, as described above.

Mice were sacrificed after measuring muscle function and GDE expression in muscles was evaluated by Western Blot in heart, diaphragm, triceps and quadriceps. Mouse tissues were homogenized in RIPA buffer (#10017003, FisherBioblock scientific) and protein concentration was determined using the BCA Protein Assay (Thermo Fisher Scientific, Waltham, MA). Seventy-five micrograms of proteins were loaded per lane. Proteins were separated by SDS-PAGE electrophoresis (4-12% gradient, NuPAGE Novex Bis-Tris Gel 1.0 mm, Life Technologies) and transferred onto a Nitrocellulose Blotting Membrane 0.2 μm (GE Healthcare). A rabbit polyclonal antibody to human C-ter part (from 1182 to 1533 aa) of GDE protein (1:1000; 16582-1-AP, Proteintech) was used as primary antibodies followed by corresponding IRDye-800CW-conjugated antibody (1:10,000) according to the manufacturer's instructions (Li-COR Biosciences). Infrared fluorescence of the secondary antibodies was visualised on an Odyssey CLX Imaging System (Li-COR Biosciences). The Precision Plus Protein Standard (Bio-Rad) was used as molecular weight marker.

Results

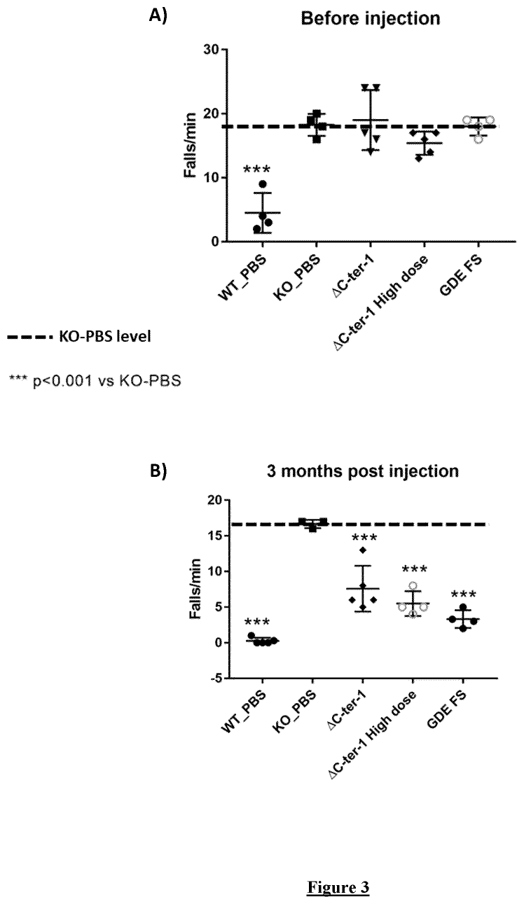

The efficacy of the C-terminal truncated GDE (ΔCter-1) was evaluated and compared to the efficacy of full length GDE for the rescue of muscle strength, 3 months after vectors injection (FIG. 3). Before injection (FIG. 3A), functional evaluation showed a similar number of falls between groups of GSDIII mice (˜20 falls per minute) and was much higher than WT mice (5 falls per minute). Three months after injection (FIG. 3B), the groups of mice treated with vectors encoding either ΔCter-1 GDE or full size GDE showed a significant functional improvement. A slight improvement was observed by increasing the dose of ΔCter-1 GDE.

Analysis of GDE expression by Western blot (FIG. 4) showed that the ΔCter-1 protein is well expressed in the different tissues.

Taken together, these data indicate a similar activity of the C-terminal truncated ΔCter-1-GDE enzyme when compared to the full-size GDE protein in muscle.

1-16. (canceled)

17. A functional truncated GDE polypeptide, wherein said functional truncated GDE polypeptide comprises a C-terminal deletion of at least 1 amino acid and at most 112 amino acids with respect to a reference functional full-length human GDE sequence, and wherein the functional truncated GDE polypeptide does not comprise the sequence as shown in SEQ ID NO:59.

18. The functional truncated GDE polypeptide of claim 17, wherein the reference functional full-length human GDE has an amino acid sequence as shown in SEQ ID NO:1, SEQ ID NO:2, SEQ ID NO:3, SEQ ID NO:61, SEQ ID NO:62 or SEQ ID NO:63, or has an amino acid sequence having at least 80, 85, 90, 95, 96, 97, 98 or at least 99 percent sequence identity to SEQ ID NO:1, SEQ ID NO:2, SEQ ID NO:3, SEQ ID NO:61, SEQ ID NO:62 or SEQ ID NO:63.

19. The functional truncated GDE polypeptide of claim 17, wherein:
(i) the reference functional full-length human GDE sequence has an amino acid sequence as shown in SEQ ID NO:1 or SEQ ID NO:61, and said truncated GDE polypeptide has a deletion of at least one amino acid and of at most 112 amino acids selected from amino acids at positions 1421 to 1532 with respect to SEQ ID NO:1 or SEQ ID NO:61;
(ii) the reference functional full-length human GDE sequence has an amino acid sequence as shown in SEQ ID NO:2 or SEQ ID NO:62, and said truncated GDE polypeptide has a deletion of at least one amino acid and of at most 112 amino acids selected from amino acids at positions 1404 to 1515 with respect to SEQ ID NO:2 or SEQ ID NO:62; or
(iii) the reference functional full-length human GDE sequence has an amino acid sequence as shown in SEQ ID NO:3 or SEQ ID NO:63, and said truncated GDE polypeptide has a deletion of at least one amino acid and of at most 112 amino acids selected from amino acids at positions 1405 to 1516 with respect to SEQ ID NO:3 or SEQ ID NO:63.

20. The functional truncated GDE polypeptide of claim 17, wherein:
(i) the reference functional full-length human GDE sequence has an amino acid sequence as shown in SEQ ID NO:1 or SEQ ID NO:61, and said truncated GDE polypeptide has a deletion of at least 5 consecutive amino acids selected from amino acids at positions 1421 to 1532 with respect to SEQ ID NO:1 or SEQ ID NO:61;
(ii) the reference functional full-length human GDE sequence has an amino acid sequence as shown in SEQ ID NO:2 or SEQ ID NO:62, and said truncated GDE polypeptide has a deletion of at least 5 consecutive amino acids selected from amino acids at positions 1404 to 1515 with respect to SEQ ID NO:2 or SEQ ID NO:62; or
(iii) the reference functional full-length human GDE sequence has an amino acid sequence as shown in SEQ ID NO:3 or SEQ ID NO:63, and said truncated GDE polypeptide has a deletion of at least 5 consecutive amino acids selected from amino acids at positions 1405 to 1516 with respect to SEQ ID NO:3 or SEQ ID NO:63.

21. The functional truncated GDE polypeptide of claim 17, wherein the functional truncated GDE polypeptide further comprises a deletion or a combination of deletions with respect to SEQ ID NO:1, SEQ ID NO:2, SEQ ID NO:3, SEQ ID NO:61, SEQ ID NO:62 or SEQ ID NO:63, wherein the deletion(s) is(are) selected from any deletion referred to as Δ1, Δ2, Δ3, Δ4, Δ5, Δ6, Δ7, Δ8, Δ9, Δ10, Δ11, Δ12, and Δ13

| | Position | Position | Position |
|---|---|---|---|
| | of the deleted | of the deleted | of the deleted |
| | amino acids with | amino acids with | amino acids with |
| | respect to | respect to | respect to |
| | SEQ ID NO: 1 or | SEQ ID NO: 2 or | SEQ ID NO: 3 or |
| Deletion | SEQ ID NO: 61 | SEQ ID NO: 62 | SEQ ID NO: 63 |
| Δ1 | 1-156 | 1-139 | 1-140 |
| Δ2 | 361-428 | 344-411 | 345-412 |
| Δ3 | 668-769 | 651-752 | 652-753 |
| Δ4 | 895-1087 | 878-1070 | 879-1071 |
| Δ5 | 223-320 | 206-303 | 207-304 |
| Δ6 | 360-428 | 343-411 | 344-412 |
| Δ7 | 669-720 | 652-703 | 653-704 |
| Δ8 | 1-280 | 1-263 | 1-264 |
| Δ9 | 1-15 | — | — |
| Δ10 | 1-30 | 1-13 | 1-14 |
| Δ11 | 1-81 | 1-64 | 1-65 |
| Δ12 | 1-103 | 1-86 | 1-87 |
| Δ13 | 1-129 | 1-112 | 1-113 |

22. The functional truncated GDE polypeptide of claim 17, wherein the functional truncated GDE polypeptide comprises SEQ ID NO:7-14, SEQ ID NO:25-33 or SEQ ID NO:64-66, or has at least 80 percent sequence identity to SEQ ID NO:7-14, SEQ ID NO:25-33 or SEQ ID NO:64-66.

23. The functional truncated GDE polypeptide of claim 17, wherein the functional truncated GDE polypeptide comprises SEQ ID NO:7, SEQ ID NO:8, SEQ ID NO:9, SEQ ID NO:64, SEQ ID NO:65 or SEQ ID NO:66 or has an amino acid sequence having at least 80 percent sequence identity to SEQ ID NO:7, SEQ ID NO:8, SEQ ID NO:9, SEQ ID NO:64, SEQ ID NO:65 or SEQ ID NO:66.

24. The functional truncated GDE polypeptide of claim 17, wherein the functional truncated GDE polypeptide has the amino acid sequence of SEQ ID NO:7 or has an amino acid sequence having at least 80 percent sequence identity to SEQ ID NO:7.

25. A nucleic acid molecule encoding the functional truncated GDE polypeptide of claim 17.

26. An expression cassette comprising:
a promoter;
optionally, an intron;
the nucleic acid molecule of claim 25; and
a polyadenylation signal.

27. A vector comprising the nucleic acid molecule of claim 25 or the expression cassette comprising said nucleic acid molecule.

28. The vector of claim 27, wherein said vector is an AAV vector.

29. An isolated cell transformed with the nucleic acid molecule of claim 25, an expression cassette comprising said nucleic acid molecule or a vector comprising said nucleic acid molecule.

30. The isolated cell of claim 29, wherein the cell is a liver cell, a muscle cell, a cardiac cell or CNS cell.

31. A method of treating a disease caused by a mutation in the AGL gene encoding GDE comprising administering a functional truncated GDE polypeptide of claim 17 to a subject having a mutation in said AGL gene.

32. The method of claim 31, wherein said disease is glycogen storage disease III (GSDIII).

33. A method of treating a disease caused by a mutation in the AGL gene encoding GDE comprising administering a nucleic acid of claim 25 or an expression cassette or vector comprising said nucleic acid to a subject having a mutation in said AGL gene.

34. The method of claim 33, wherein said disease is glycogen storage disease III (GSDIII).

35. A method of treating a disease caused by a mutation in the AGL gene encoding GDE comprising administering a cell of claim 29 to a subject having a mutation in said AGL gene.

36. The method of claim 35, wherein said disease is glycogen storage disease III (GSDIII).


SEQUENCE LISTING (67 sequences)

SEQ ID NO: 1 - protein, 1532 aa, artificial
  GDE human isoform 1 (variant)
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 2 - protein, 1515 aa, artificial
  GDE Human Isoform 5 (variant)
MSLLTCAFYLGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 3 - protein, 1516 aa, artificial
  GDE human isoform 6 (variant)
MAPILSINLFIGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 4 - DNA, 4599 bp, artificial
  GDE isoform 1 coding sequence (variant)
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaacgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 5 - DNA, 4599 bp, artificial
  GDEco1
atgggccatagtaaacagattcgcatactcctcttgaacgagatggagaaactggagaagacattgtttcggttggagcaggggtacgagctccagtttcgcctgggaccgacgctccaaggcaaagctgtgactgtatacacgaactatccattcccgggggagacgtttaacagggagaagtttaggtccctggactgggagaacccaaccgaacgagaggacgattccgataaatattgcaagctcaacttgcagcaaagtggcagctttcaatattactttctccaaggcaatgagaaaagtgggggggggtatattgttgtcgatccaatactgcgcgtaggggcagataatcacgttctcccgctggattgcgtcactctccagacattcttggctaaatgcttggggccgtttgatgaatgggagtctcgcttgcgagtggccaaagagtcaggttataacatgattcacttcacaccactccagacattgggacttagtcggagctgttactcactcgcaaatcaattggagcttaacccagacttcagtcggccaaatcggaagtacacgtggaacgacgttggacaacttgtcgaaaagttgaagaaagagtggaatgtgatttgcatcactgacgtggtgtacaatcacaccgcagccaacagcaagtggattcaggagcacccagagtgtgcgtacaacctggtgaactcacctcacctcaaacccgcctgggtgctcgatagggctttgtggcgcttttcttgcgacgtagcggaaggaaagtataaagagaaaggaatacccgccctcatagaaaacgatcatcacatgaattctatacggaaaatcatctgggaggatatatttccgaaacttaaactttgggagttctttcaagtagatgtaaacaaggcggtggagcaattcaggaggctcctcacccaagagaatcgccgggttactaaatctgacccgaatcaacaccttacaataatccaagatccggaatacaggaggtttggttgcactgtcgatatgaatattgcgcttactacgttcatcccccacgacaagggcccggccgcaatagaagaatgctgcaattggttccacaagcggatggaagaactgaactctgaaaagcaccgccttataaattatcaccaagagcaggctgtgaactgtctgctcggtaacgttttttacgagcgcctggccggacacggacctaaactcgggccagtcactcgaaaacacccactggttacgcgatacttcacattcccgttcgaggagatcgacttttctatggaggaatctatgatccacctcccaaataaagcttgttttcttatggcgcacaacggatgggttatgggggacgacccactgcgaaacttcgcagaaccgggtagtgaggtctaccttaggcgcgagctcatttgttggggcgacagcgtcaagctccggtatggaaataagccagaggattgcccttacttgtgggcacacatgaagaagtatacggaaataacagctacctacttccagggggtacgactggataactgccactccacaccgttgcacgtggccgagtatatgctcgacgctgcgcgcaatttgcagccaaatctgtacgtcgtggcagagcttttcactggaagtgaggacttggataacgtctttgtgactcgcctgggaattagtagcttgataagggaggctatgtccgcgtacaacagtcacgaggaaggacgattggtttatcgatatgggggcgagcctgtaggctcctttgtgcaaccctgcttgcggccccttatgcccgctatagcacacgcgctcttcatggatatcacgcacgataatgaatgccccatagtacacagatccgcctacgacgcccttccatctacgacaatcgtctctatggcctgctgcgcctccggcagcactagaggctacgacgaactcgtcccacaccagatttcagtggtatcagaggaacggttttacactaaatggaaccctgaggcgctcccatctaatactggcgaagtaaatttccagtccggaatcattgcggcccgctgtgctatctccaagttgcatcaggaacttggagctaaaggtttcattcaagtatatgtcgatcaggtcgatgaagatattgtggctgtgacccgacactccccatcaattcatcaaagtgtagtggctgtaactcggacggcttttcgcaacccaaagacttcattctactccaaagaggttccacagatgtgtattccgggaaagatagaagaagtggtattggaagcccggaccatcgagaggaacactaaaccatatcgaaaagacgagaactccattaacggaacccctgacatcactgttgagatccgcgagcatattcagcttaacgaaagcaaaatcgttaagcaggccggcgttgccactaagggaccaaacgaatatatccaagaaatcgaattcgaaaacctcagtcctggctccgttattatctttcgcgtatccctcgacccacacgcccaagttgcggtagggatcttgagaaaccacctcacacagttcagcccacactttaaatcaggctccctcgccgttgataacgcggacccaatacttaagatcccctttgcatcccttgcgtatcgacttactctcgcagagcttaatcaaatattgtaccgctgcgagtccgaagagaaggaagacggtggtggctgctacgacatccctaattggagtgcacttaagtacgcggggctgcagggactgatgtcagtgcttgcagagataaggccgaagaatgaccttggccatccattttgtaataatctccgaagtggtgattggatgatagattacgtatcaaaccgcttgatcagtcggtctggtaccatcgcggaagtgggaaagtggttgcaggcaatgttcttttatctcaaacaaatcccacggtacttgataccttgctatttcgacgcaattctcatcggtgcatacacgaccttgctggacacggcctggaagcagatgtctagcttcgttcagaacggttctaccttcgtaaagcacctctcattgggttcagtccaactctgcggagtcgggaaattcccttcacttcctattctctcacctgccctcatggacgtgccctaccggctgaacgaaattactaaggagaaggaacagtgttgtgtttctttggcggcaggcttgccgcacttttccagtggaatcttcagatgttggggacgggacacattcattgcgctccggggtattttgttgataacgggccgatacgttgaggcacgaaatattattctggcattcgccgggaccttgcggcacgggctgatacccaacctgctgggcgaagggatttacgctcgctataactgccgagacgcagtttggtggtggctgcagtgtattcaggactattgtaagatggtaccgaacgggctcgacatcttgaagtgtcccgttagtcgaatgtatcccaccgacgattcagctcccctgcccgcgggaacacttgaccaaccactctttgaagtgatccaagaggctatgcagaaacacatgcagggaatacagttccgagaacgaaacgcagggccgcagattgatcgaaatatgaaagacgaaggatttaatatcacggcaggggtcgacgaagagacggggtttgtctacggcgggaatagatttaactgcggcacctggatggataaaatgggagaaagtgaccgagcacggaaccggggcataccagcaaccccccgagacgggagcgctgttgagatcgtgggtctgtctaagagtgcggttcgctggcttctcgagctttcaaaaaaaaatatatttccttaccacgaggtcacggtcaaaaggcatggaaaagccataaaagtgtcatacgacgagtggaataggaaaatacaagataactttgaaaagctgtttcacgttagcgaagatcccagcgatctcaacgaaaaacatcccaatctggttcacaaacgcgggatctataaagactcatatggagctagttctccttggtgcgattatcaactgagaccgaactttacaatcgccatggtagttgcgcccgagctctttactacagaaaaagcctggaaggcacttgagattgcggaaaagaaactgcttggccctctcgggatgaaaacgcttgatcccgacgacatggtctattgcgggatttacgacaacgcattggacaacgacaactacaacttggcgaaaggatttaattatcaccagggtcctgagtggttgtggcccattggatactttcttcgagcgaagctgtatttttcaaggctgatggggccggaaacgacagcgaaaactattgtgcttgtcaaaaacgtgcttagcaggcattacgtgcacctcgagcgcagcccttggaaaggacttccggagcttacgaacgaaaacgcccagtattgtccatttagctgtgagacgcaggcctggtctattgctaccatcctcgagacactctacgacttgtag

SEQ ID NO: 6 - DNA, 4599 bp, artificial
  GDEco2
atgggccacagcaagcagatcagaatcctgctgctgaacgagatggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga

SEQ ID NO: 7 - protein, 1420 aa, artificial
  Delta Cter-1 - GDE isoform 1 deleted of 112 AA
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 8 - protein, 1403 aa, artificial
  Delta Cter-2 - GDE isoform 2 deleted of 112 AA
MSLLTCAFYLGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 9 - protein, 1404 aa, artificial
  Delta Cter-3 - GDE isoform 3 deleted of 112 AA
MAPILSINLFIGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 10 - protein, 1406 aa, artificial
  Delta N-ter (1a)  +  Delta Cter-1
MEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 11 - protein, 1391 aa, artificial
  Delta Nter (1b1)  +  Delta Cter-1
MLQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 12 - protein, 1340 aa, artificial
  Delta Nter (1b2)  +  Delta Cter-1
MKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 13 - protein, 1318 aa, artificial
  Delta Nter (1b3)  +  Delta Cter-1
MGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 14 - protein, 1292 aa, artificial
  Delta Nter (1c)  +  Delta Cter-1
MQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 15 - DNA, 4221 bp, artificial
  Delta Nter (1a) + Delta Cter-1 (codon optimized)
atggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 16 - DNA, 4176 bp, artificial
  Delta Nter (1b1) + Delta Cter-1 (codon optimized)
atgctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 17 - DNA, 4023 bp, artificial
  Delta N-ter (1b2) + Delta Cter-1 (codon optimized)
atgaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 18 - DNA, 3957 bp, artificial
  Delta Nter (1b3) + Delta Cter-1 (codon optimized)
atgggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 19 - DNA, 3879 bp, artificial
  Delta Nter (1c) + Delta Cter-1 (codon optimized)
atgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 20 - DNA, 4221 bp, artificial
  Delta Nter (1a) + Delta Cter-1 (not codon optimized)
atggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 21 - DNA, 4176 bp, artificial
  Delta Nter (1b1) + Delta Cter-1 (not codon optimized)
atgctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 22 - DNA, 4023 bp, artificial
  Delta Nter (1b2) + Delta Cter-1 (not codon optimized)
atgaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 23 - DNA, 3957 bp, artificial
  Delta Nter (1b3) + Delta Cter-1 (not codon optimized)
atgggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 24 - DNA, 3879 bp, artificial
  Delta Nter (1c) + Delta Cter-1 (not codon optimized)
atgcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 25 - protein, 1264 aa, artificial
  Delta 1 + Delta Cter-1
MIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 26 - protein, 1352 aa, artificial
  Delta 2 + Delta Cter-1
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 27 - protein, 1318 aa, artificial
  Delta 3 + Delta Cter-1
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 28 - protein, 1250 aa, artificial
  Delta 2,3 + Delta Cter-1
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 29 - protein, 1227 aa, artificial
  Delta 4 + Delta Cter-1
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 30 - protein, 1201 aa, artificial
  Delta 5 + delta Cter-1
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTANRRVTKSDPNQHLTIIQDPEYRRFGCTVDMNIALTTFIPYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQIYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 31 - protein, 1140 aa, artificial
  Delta 6 + Delta Cter-1
MNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 32 - protein, 996 aa, artificial
  Delta 7 + Delta Cter-1
MVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 33 - protein, 1191 aa, artificial
  Delta 8 + Delta Cter-1
MHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 34 - DNA, 3795 bp, artificial
  Delta 1 + Delta Cter-1
atgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 35 - DNA, 3795 bp, artificial
  Delta 1 + Delta Cter-1 (codon optimized)
atgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 36 - DNA, 4059 bp, artificial
  Delta 2 + Delta Cter-1
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacattattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 37 - DNA, 3957 bp, artificial
  Delta 3 + Delta Cter-1
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 38 - DNA, 3753 bp, artificial
  Delta 2,3 + Delta Cter-1
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacattattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 39 - DNA, 3753 bp, artificial
  Delta 2,3 + Delta Cter-1 (codon optimized)
atgggccacagcaagcagatcagaatcctgctgctgaacgagatggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccactacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 40 - DNA, 3684 bp, artificial
  Delta 4 + Delta Cter-1
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 41 - DNA, 3684 bp, artificial
  Delta 4 + Delta Cter-1 (codon optimized)
atgggccacagcaagcagatcagaatcctgctgctgaacgagatggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactga

SEQ ID NO: 42 - DNA, 3606 bp, artificial
  Delta 5 + Delta Cter-1
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctaataggcgagtaaccaagtctgatccaaaccaacaccttacgattattcaagatcctgaatacagacggtttggctgtactgtagatatgaacattgcactaacgactttcataccatattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 43 - DNA, 3423 bp, artificial
  Delta 6 + Delta Cter-1
atgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 44 - DNA, 2991 bp, artificial
  Delta 7 + Delta Cter-1
atggttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 45 - DNA, 3576 bp, artificial
  Delta 8 + Delta Cter-1
atgcatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 46 - DNA, 441 bp, artificial
  HBB2 intron
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 47 - DNA, 441 bp, artificial
  modified HBB2 intron
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 48 - DNA, 1438 bp, artificial
  FIX intron
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctatgtcaaacatcttggagttgatatttggggaaacacaatactcagttgagttccctaggggagaaaagcacgcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttca

SEQ ID NO: 49 - DNA, 1438 bp, artificial
  Modified FIX intron
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacattgcttttgttgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaattgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctttgtcaaacatcttggagttgatatttggggaaacacaatactcagttgagttccctaggggagaaaagcacgcttaagaattgacataaagagtaggaagttagctattgcaacatatatcactttgttttttcacaactacagtgactttttgtatttcccagaggaaggcatacagggaagaaattatcccatttggacaaacagcttgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgttggatcactttgcaaagatcctcattgagctattttcaagtgttgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcttgttgttttcttttttgctaaaactaaagaattattcttttacatttca

SEQ ID NO: 50 - DNA, 881 bp, artificial
  Chicken beta-globin intron
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctc

SEQ ID NO: 51 - DNA, 881 bp, artificial
  modified chicken-beta globin intron
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttttggtaatcgtgcgagagggcgcagggacttcctttgtcccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctc

SEQ ID NO: 52 - DNA, 17 bp, artificial
  sNRP1 polyadenylation signal
aaataaaatacgaaatg

SEQ ID NO: 53 - DNA, 169 bp, artificial
  bGH polyadenylation signal
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 54 - DNA, 58 bp, artificial
  pA58 polyadenylation signal
gcggccgcaataaaagatctttattttcattagatctgtgtgttggttttttgtgtgt

SEQ ID NO: 55 - DNA, 5124 bp, artificial
  Expression cassette (ITR + miniCMV + GDE Delta Cter-1 + bGH polyA)
gcgcgctcgctcgctcactgaggccgcccgggcaaagcccgggcgtcgggcgacctttggtcgcccggcctcagtgagcgagcgagcgcgcagagagggagtggccaactccatcactaggggttccttctagataacctaggactagtgcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgttaattaagctagcacgcgtcgacatgcatgatatcctgcagctagctcgagagatctgccaccatgggccacagcaagcagatcagaatcctgctgctgaacgagatggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgaccagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctacagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgactgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctagaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 56 - DNA, 4263 bp, artificial
  Delta Cter-1
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtaaattctccacacttaaaacctgcctgggtcttagacagagcactttggcgtttctcctgtgatgttgcagaagggaaatacaaagaaaagggaatacctgctttgattgaaaatgatcaccatatgaactccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaggacctagacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtaactagaactgctttcaggaatcccaagacttcattttacagcaaggaagtgcctcaaatgtgcatccctggcaaaattgaagaagtagttcttgaagctagaactattgagagaaacacgaaaccttataggaaggatgaaaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggcattcttcgaaatcatctgacacaattcagtcctcactttaaatctggcagcctagctgttgacaatgcagatcctatattaaaaattccttttgcttctcttgcctatagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgaggtctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggactagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 57 - DNA, 4215 bp, artificial
  Delta Cter-3
atggccccaattctgagcattaatttatttattgggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagattag

SEQ ID NO: 58 - DNA, 4599 bp, Homo sapiens
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 59 - protein, 15 aa, artificial
  GDE Amino acids mutated in GSD patient
HFAVIWFTVEFMTMH

SEQ ID NO: 60 - protein, 16 aa, Homo sapiens
DMVYCGIYDNALDNDN

SEQ ID NO: 61 - protein, 1532 aa, Homo sapiens
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 62 - protein, 1515 aa, Homo sapiens
MSLLTCAFYLGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 63 - protein, 1516 aa, Homo sapiens
MAPILSINLFIGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 64 - protein, 1420 aa, artificial
  WT GDE isoform 1 + Delta Cter-1
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 65 - protein, 1403 aa, artificial
  WT GDE isoform 5 + Delta Cter-2
MSLLTCAFYLGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 66 - protein, 1404 aa, artificial
  WT GDE isoform 6 + Delta Cter-3
MAPILSINLFIGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPD

SEQ ID NO: 67 - DNA, 4599 bp, artificial
  GDE co2 (encoding WT isoform 1)
atgggccacagcaagcagatcagaatcctgctgctgaacgagatggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga